(19) United States
(12) Patent Application Publication
(10) Pub. No.: US 2010/0174104 A1
(43) Pub. Date: Jul. 8, 2010

(54) COBALT-CATALYZED ASYMMETRIC CYCLOPROPANATION WITH DIAZOSULFONES

(76) Inventors: X. Peter Zhang, Tampa, FL (US); Shifa Zhu, Tampa, FL (US)

Correspondence Address: BRYAN CAVE LLP, 211 NORTH BROADWAY, SUITE 3600, ST. LOUIS, MO, 63102-2750, US

(21) Appl. No.: 12/404,435

(22) Filed: Mar. 16, 2009

Related U.S. Application Data
(60) Provisional application No. 61/036,807, filed on Mar. 14, 2008.
(60) Provisional application No. 61/038,655, filed on Mar. 21, 2008.

(51) Int. Cl. C07C 255/46; C07C 315/00; C07C 201/06

(52) U.S. Cl. 558/426; 568/34; 568/33; 564/441; 560/11

(57) ABSTRACT

Asymmetric cyclopropanation of olefins with diazosulfones.

### CROSS-REFERENCE TO RELATED APPLICATIONS

This application claims priority to U.S. Provisional Application Ser. No. 61/036,807, filed Mar. 14, 2008, and U.S. Provisional Application Ser. No. 61/038,655, filed Mar. 21, 2008, both of which are incorporated herein by reference in their entireties.

### STATEMENT OF GOVERNMENT RIGHTS

This invention was made with Government support under grant number NSF #0711024, awarded by the National Science Foundation, Division of Chemistry, and under grant number CRIF: MU-0443611, awarded by the National Science Foundation. The Government has certain rights in the invention.

### FIELD OF THE INVENTION

The present invention generally relates to metal-catalyzed cyclopropanation of olefins. More particularly, the present invention relates to a process for asymmetric cyclopropanation of olefins in the presence of a diazosulfone reagent. Even more particularly, the present invention relates to a process for asymmetric cyclopropanation of olefins in the presence of a diazosulfone reagent using a cobalt porphyrin complex.

### BACKGROUND

Cyclopropane derivatives are a unique class of compounds with fundamental importance of being the smallest all-carbon cyclic molecules as well as having practical significance as recurring units in numerous natural products and as valuable synthons for many chemical transformations. (Pietruszka, J., Chem. Rev. 2003, 103, 1051; Wessjohann et al., Chem. Rev. 2003, 103, 1625; Donaldson, W. A., Chem. Rev. Tetrahedron 2001, 57, 8589; Salaun, J., Chem. Rev. 1989, 89, 1247.) Of different methods, metal-catalyzed cyclopropanation of alkenes with diazo reagents is considered one of the most versatile methods for the stereoselective construction of the three-membered ring structures. (Lebel et al., Chem. Rev. 2003, 103, 977; Davies et al., Org. React. 2001, 57, 1; Doyle et al., Chem. Rev. 1998, 98, 911; Padwa et al., Tetrahedron 1992, 48, 5385.) Among known catalytic systems, Cu-, Rh-, and Ru-catalyzed asymmetric processes have been successfully developed to permit olefin cyclopropanation in high yields and high selectivities. (Fritschi et al., Agnew. Chem., Int. Ed. Engl. 1986, 25, 1005; Evans et al., J. Am. Chem. Soc. 1991, 113, 726; Lo et al., J. Am. Chem. Soc. 1998, 120, 10270; Maxwell et al., Organometallics 1992, 11, 645; Doyle et al., J. Am. Chem. Soc. 1993, 115, 9968; Davies et al., J. Am. Chem. Soc. 1996; 118; 6897; Nishiyama et al., J. Am. Chem. Soc. 1994, 116, 2223; Che et al., J. Am. Chem. Soc. 2001, 123, 4119.) While the vast majority of those catalytic systems employed diazocarbonyls, mostly diazoacetates, as carbene sources, metal-catalyzed asymmetric cyclopropanation reactions with other types of diazo reagents are underdeveloped.

Following our original discovery of cobalt porphyrin [Co(Por)]'s unique catalytic capability for cyclopropanation, a family of D₂-symmetrical chiral porphyrins was designed and synthesized via a versatile, modular approach for the development of the asymmetric variant of the Co-catalyzed process. (Huang et al., J. Org. Chem. 2003, 68, 8179; Chen et al., J. Am. Chem. Soc., 2004, 126, 14718; For non-porphyrin Co-catalyzed Cyclopropanation systems, see: Niimi et al., Adv. Synth. Catal. 2001, 343, 79; Niimi et al., Tetrahedron Lett. 2000, 41, 3647; Ikeno et al., Synlett 2001, 406; Nakamura et al., J. Am. Chem. Soc. 1978, 100, 3443.) Among them, [Co(P1)] has proved to be one of the most selective catalysts for asymmetric cyclopropanation of both electron-sufficient (styrene derivatives) and electron-deficient (α,β-unsaturated carbonyls and nitriles) olefins with diazoacetates. (Chen et al., J. Org. Chem. 2007, 72, 5931; Chen et al., J. Am. Chem. Soc. 2007, 129, 12074.) To further augment its substrate generality, we decided to explore the effectiveness of the Co-based catalytic system for asymmetric cyclopropanation with diazo reagents, rather than diazoacetates. As a result of this effort, we wish to describe herein a highly effective catalytic system for asymmetric cyclopropanation employing diazosulfones. This is a class of known diazo reagents that has not been previously employed for asymmetric cyclopropanation except via a Cu-based intramolecular system reported by Nakada and co-workers. (Honma et al., J. Am. Chem. Soc. 2003, 125, 2860; Sawada et al., Adv. Synth. Catal. 2005, 347, 1527; Takeda et al., Tetrahedron; Asymmetry 2006, 17, 2896; For a Rh-based earlier attempt of intramolecular asymmetric cyclopropanation, see: Kennedy et al., J. Chem. Soc., Chem. Commun. 1990, 361; For a Cu-catalyzed asymmetric intermolecular cyclopropanation with α-diazosulfonate esters, see: Ye et al., New J. Chem. 2005, 29, 1159; For a Rh-catalyzed asymmetric cyclopropanation of acetylenes with tosyldiazomethane, see: Weatherhead-Kloster et al., Org. Lett. 2006, 8, 171.) Asymmetric olefin cyclopropanation with diazosulfones would be highly desirable as the resulting cyclopropyl sulfones have found a variety of applications. (For select examples of other approaches for the syntheses and applications of optically active cyclopropyl sulfones, see: Ruano et al., Org. Lett. 2004, 6, 4945; Bernard et al., Org. Lett. 2005, 7, 4565; Midura et al., Eur. J. Org. Chem. 2005, 653; Das et al., J. Org. Chem. 2007, 72, 9181. For an interesting synthesis of cyclopropyl sulfones via Rh-catalyzed intramolecular 1,3 C—H carbene insertion, see: Shi et al., Org. Lett. 2005, 7, 3103.)

### SUMMARY OF THE INVENTION

Among the various aspects of the present invention, therefore, is a process for the asymmetric cyclopropanation of olefins with diazosulfones and the sulfone-substituted cyclopropanes resulting therefrom.

In one embodiment, the present invention is directed to a process for the preparation of sulfone-substituted cyclopropanes. The process comprises treating an alkene with a diazosulfone in the presence of a metal porphyrin complex.

In one embodiment, the present invention is directed to a process for the preparation of sulfone-substituted cyclopropanes. The process comprises treating an alkene with a diazosulfone in the presence of a cobalt complex.

The present invention is further directed to a process for asymmetric cyclopropanation of an olefin, the process comprising treating the olefin with a diazosulfone reagent in the presence of a cobalt complex of a D₂-symmetric chiral porphyrin.

In another embodiment, the present invention is directed to sulfone substituted cyclopropanes having the structure:

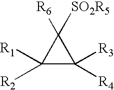

wherein R₁, R₂, R₃, R₄, are independently hydrogen, hydrocarbyl, substituted hydrocarbyl, heterocyclo, or EWG (electron-withdrawing group), and R₅ and R₆ are independently hydrogen, hydrocarbyl, substituted hydrocarbyl, or heterocyclo.

In another embodiment, the present invention is directed to sulfone substituted cyclopropanes having the structure:

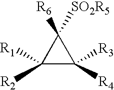

wherein R₁, R₂, R₃, R₄, R₅, and R₆ are independently hydrogen, hydrocarbyl or substituted hydrocarbyl.

Other aspects of the invention will be in part apparent, and in part pointed out hereinafter.

### BRIEF DESCRIPTION OF THE DRAWINGS

For a fuller understanding of the invention, reference should be made to the following detailed description, taken in connection with the accompanying drawings, in which:

FIG. 1 shows structures of D₂-symmetric chiral porphyrins.

FIG. 2 shows the X-ray structure of P6 (porphyrin 6), indicating hydrogen bonding interactions.

FIG. 3 shows the three-dimensional structure for porphyrin [H₂(P6)].

FIG. 4 shows the three-dimensional structure for 1-methyl-4-(2-phenylcyclopropylsulfonyl)benzene.

### DETAILED DESCRIPTION OF THE PREFERRED EMBODIMENTS

In general, the process of the present invention may be used to form a wide range of sulfone-substituted cyclopropanes. In this process, any of a wide range of alkenes are treated with any of a wide range of diazosulfones in the presence of a metal porphyrin catalyst.

### Olefins

In general, the alkene, also referred to herein as an olefin, may be any of a wide range of olefins. In one embodiment, the olefin corresponds to Formula 1:

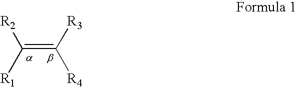

wherein R₁ and R₂ are substituents of the α-carbon of the ethylenic bond, and R₃ and R₄ are substituents of the β-carbon of the ethylenic bond. R₁, R₂, R₃, and R₄ are independently hydrogen, hydrocarbyl, substituted hydrocarbyl, heterocyclo, or EWG (electron-withdrawing group). In one embodiment, R₁ is hydrogen. In another embodiment, R₁ is alkyl or substituted alkyl. In one embodiment, R₂ is hydrogen. In another embodiment, R₂ is alkyl or substituted alkyl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, two of R₁, R₂, R₃ and R₄ are hydrogen. In another embodiment, three of R₁, R₂, R₃, and R₄ are hydrogen. In one embodiment, R₁, R₂ and the α-carbon, or R₃, R₄ and the β-carbon, form a carbocyclic or heterocyclic ring. In another embodiment, R₁, R₃, the α-carbon, and the β-carbon, or R₂, R₄, the α-carbon, and the β-carbon form a carbocyclic or heterocyclic ring. In another embodiment, R₁, R₄, the α-carbon, and the β-carbon, or R₂, R₃, the α-carbon, and the β-carbon form a carbocyclic or heterocyclic ring. In one preferred embodiment, at least one of R₁, R₂, R₃, and R₄ is alkyl, aryl, substituted phenyl, —CN, —C(O)R₂₂, or —C(O)OR₂₂ wherein R₂₂ is hydrogen, optionally substituted alkyl, optionally substituted alkenyl, optionally substituted alkynyl, or optionally substituted aryl. In a further preferred embodiment, at least one of R₁, R₂, R₃ and R₄ is —CN, —C(O)CH₃, —C(O)OCH₃, or —C(O)OC₂H₅. In another preferred embodiment, at least one of R₁, R₂, R₃ and R₄ is phenyl, tert-butyl phenyl, methoxyphenyl, trifluoromethyl phenyl, nitrophenyl, or naphthyl. In a further preferred embodiment, at least one of R₁, R₂, R₃ and R₄ is phenyl, p-tert-butyl phenyl, p-methoxyphenyl, p-trifluoromethyl phenyl, 3-nitrophenyl, or naphthyl. In certain preferred embodiments, the olefin is an aromatic olefin, an α,β-unsaturated ester, an α,β-unsaturated ketone, or an α,β-unsaturated nitrile.

In one embodiment, the alkene is selected from the group consisting of aromatic alkenes, non-aromatic alkenes, di-substituted alkenes, tri-substituted alkenes, tetra-substituted alkenes, cis-alkenes, trans-alkenes, cyclic alkenes, and non-cyclic alkenes. In one preferred embodiment, the alkene corresponds to the following structure:

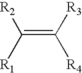

wherein R₁, R₂, R₃, and R₄ are independently hydrogen, hydrocarbyl or substituted hydrocarbyl. For cyclic alkenes, two of R₁, R₂, R₃, and R₄, in combination with the atoms of the alkene to which they are attached define a ring. In one embodiment, R₁, R₂, R₃, and R₄ are independently hydrogen, alkyl, alkenyl, alkynyl, or aryl. In a further embodiment, at least one of R₁, R₂, R₃, and R₄ is heterosubstituted and the remainder are independently hydrogen, alkyl, alkenyl, alkynyl, or aryl. In one embodiment, the alkene is an α,β-unsaturated alkene. For example, the alkene may be an α,β-unsaturated alkene, an α,β-unsaturated ester, α,β-unsaturated nitrile or α,β-unsaturated ketoalkene. In one preferred embodiment, the alkene is an α,β-unsaturated alkene corresponding to the structure:

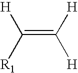

wherein R₁ is optionally substituted alkyl, optionally substituted alkenyl, optionally substituted alkynyl, or optionally substituted aryl. In one particularly preferred embodiment, R₁ is alkyl, aryl, substituted phenyl, —CN, —C(O)R₂₂, or —C(O)OR₂₂ wherein R₂₂ is optionally substituted alkyl, optionally substituted alkenyl, optionally substituted optionally substituted alkynyl, or optionally substituted aryl.

When the olefin corresponds to Formula 1 and one of R₁, R₂, R₃, and R₄ is hydrogen, e.g., R₂ is hydrogen, the olefin corresponds to Formula 2:

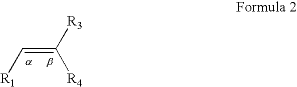

wherein R₁ is a substituent of the α-carbon of the ethylenic bond, and R₃ and R₄ are substituents of the β-carbon of the ethylenic bond, and wherein R₁, R₃, and R₄ are independently hydrogen, hydrocarbyl, substituted hydrocarbyl, or heterocyclo. In one embodiment, R₁ is hydrogen. In another embodiment, R₁ is alkyl or substituted alkyl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, two of R₁, R₃ and R₄ are hydrogen. In one embodiment, R₃, R₄ and the β-carbon form a carbocyclic or heterocyclic ring. In another embodiment, R₁, R₃, the α-carbon, and the β-carbon form a carbocyclic or heterocyclic ring. In another embodiment, R₁, R₄, the α-carbon, and the β-carbon form a carbocyclic or heterocyclic ring. In one preferred embodiment, at least one of R₁, R₃, and R₄ is alkyl, aryl, phenyl, substituted phenyl, —CN, —C(O)R₂₂, or —C(O)OR₂₂ wherein R₂₂ is optionally substituted alkyl, optionally substituted alkenyl, optionally substituted alkynyl, or optionally substituted aryl. In a further preferred embodiment, at least one of R₁, R₃ and R₄ is —CN, —C(O)CH₃, —C(O)OCH₃, or —C(O)OC₂H₅. In one embodiment, the olefin is an aromatic olefin. In another particularly preferred embodiment, at least one of R₁, R₃ and R₄ is phenyl, tert-butyl phenyl, methoxyphenyl, trifluoromethyl phenyl, nitrophenyl, or naphthyl. In a further preferred embodiment, at least one of R₁, R₃ and R₄ is phenyl, p-tert-butyl phenyl, p-methoxyphenyl, p-trifluoromethyl phenyl, 3-nitrophenyl, or naphthyl. In preferred embodiments, the olefin is an α,β-unsaturated ester, an α,β-unsaturated ketone, or an α,β-unsaturated nitrile.

When the olefin corresponds to Formula 1, R₂ is hydrogen, and one of R₃ and R₄ is hydrogen, the olefin corresponds to Formula 3-cis or Formula 3-trans:

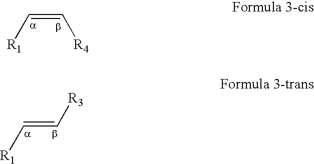

wherein R₁ is a substituent of the α-carbon of the ethylenic bond, and R₃ and R₄ are individually substituents of the β-carbon of the ethylenic bond. R₁, R₃ and R₄ are independently hydrogen, hydrocarbyl, substituted hydrocarbyl, or heterocyclo. In one embodiment, R₁ is hydrogen. In another embodiment, R₁ is alkyl or substituted alkyl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, R₁, R₃, the α-carbon, and the β-carbon form a carbocyclic or heterocyclic ring. In another embodiment, R₁, R₄, the α-carbon, and the β-carbon form a carbocyclic or heterocyclic ring. In one preferred embodiment, at least one of R₁, R₃, and R₄ is alkyl, alkenyl, aryl, heterocyclo, phenyl, substituted phenyl, —CN, —C(O)R₂₂, or —C(O)OR₂₂ wherein R₂₂ is optionally substituted alkyl, optionally substituted alkenyl, optionally substituted alkynyl, or optionally substituted aryl. In a further preferred embodiment, at least one of R₁, R₃ and R₄ is —CN, —C(O)CH₃, —C(O)OCH₃, or —C(O)OC₂H₅. In a preferred embodiment, the olefin is an aromatic olefin. In another preferred embodiment, at least one of R₁, R₃ and R₄ is phenyl, tert-butyl phenyl, methoxyphenyl, trifluoromethyl phenyl, nitrophenyl, or naphthyl. In a further preferred embodiment, at least one of R₁, R₃ and R₄ is phenyl, p-tert-butyl phenyl, p-methoxyphenyl, p-trifluoromethyl phenyl, 3-nitrophenyl, or naphthyl. In preferred embodiments, the olefin is an α,β-unsaturated ester, an (4-unsaturated ketone, or an α,β-unsaturated nitrile.

When the olefin corresponds to Formula 1 and two of the substituents on the same ethylenic carbon, e.g., R₁ and R₂, are both hydrogen, the olefin is a terminal alkene, corresponding to Formula 4:

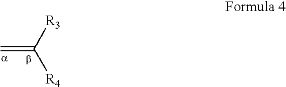

wherein R₃ and R₄ are substituents of the β-carbon of the ethylenic bond and wherein R₃ and R₄ are independently hydrogen, hydrocarbyl, substituted hydrocarbyl, or heterocyclo. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, R₃, R₄, and the β-carbon form a carbocyclic or heterocyclic ring. In one preferred embodiment, at least one of R₃ and R₄ is alkyl, alkenyl, aryl, heterocyclo, phenyl, substituted phenyl, —CN, —C(O)R₂₂, or —C(O)OR₂₂ wherein R₂₂ is optionally substituted alkyl, optionally substituted alkenyl, optionally substituted alkynyl, or optionally substituted aryl. In a further preferred embodiment, at least one of R₃ and R₄ is —CN, —C(O)CH₃, —C(O)OCH₃, or —C(O)OC₂H₅. In another preferred embodiment, at least one of R₃ and R₄ is phenyl, tert-butyl phenyl, methoxyphenyl, trifluoromethyl phenyl, nitrophenyl, or naphthyl. In a further preferred embodiment, at least one of R₃ and R₄ is phenyl, p-tert-butyl phenyl, p-methoxyphenyl, p-trifluoromethyl phenyl, 3-nitrophenyl, or naphthyl. In a preferred embodiment, the olefin is an aromatic olefin, an α,β-unsaturated ester, an α,β-unsaturated ketone, or an α,β-unsaturated nitrile.

When the olefin corresponds to Formula 1 and three of R₁, R₂, R₃, and R₄ are hydrogen, e.g., R₁, R₂, and R₃ are hydrogen, the olefin is a terminal olefin corresponding to Formula 5:

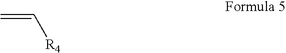

wherein R₄ is hydrogen, hydrocarbyl, substituted hydrocarbyl, or heterocyclo. In one preferred embodiment, R₄ is phenyl or substituted phenyl. In another embodiment, R₄ is alkyl or substituted alkyl. In one preferred embodiment, R₄ is —CN, —C(O)R₂₂, or —C(O)OR₂₂ wherein R₂₂ is optionally substituted alkyl, optionally substituted alkenyl, optionally substituted alkynyl, or optionally substituted aryl. In a further preferred embodiment, R₄ is —CN, —C(O)CH₃, —C(O)OCH₃, or —C(O)OC₂H₅. In another preferred embodiment, R₄ is phenyl, tert-butyl phenyl, methoxyphenyl, trifluoromethyl phenyl, nitrophenyl, or naphthyl. In a further preferred embodiment, R₄ is phenyl, p-tert-butyl phenyl, p-methoxyphenyl, p-trifluoromethyl phenyl, 3-nitrophenyl, or naphthyl. In a preferred embodiment, the olefin is an aromatic olefin, an α,β-unsaturated ester, an α,β-unsaturated ketone, or an α,β-unsaturated nitrile.

In accordance with an embodiment of the present invention, olefins possessing an electron-deficient substituent on at least one of the ethylenic carbons are cyclopropanated with a diazosulfone. In general, the olefin may be any of a wide range of olefins possessing an electron-deficient substituent on one, or both, of the ethylenic carbons. One such preferred class of olefins is α,β-unsaturated olefins possessing an electron-withdrawing substituent on the α-ethylenic carbon, the β-ethylenic carbon, or both. In one embodiment, therefore, the α-ethylenic carbon possesses an electron withdrawing substituent but the β-ethylenic carbon does not; similarly but in another embodiment, the β-ethylenic carbon possesses an electron withdrawing substituent but the α-ethylenic carbon does not. In another embodiment, the α-ethylenic carbon and the β-ethylenic carbon each possess an electron-withdrawing substituent. When the α-ethylenic carbon and the β-ethylenic carbon each possess an electron-withdrawing substituent, the electron-withdrawing substituents may be in the cis-conformation or the trans-conformation, and the electron withdrawing substituents may be the same or different.

When the olefin corresponds to Formula 1 and one but only one of R₁, R₂, R₃, and R₄ is an electron withdrawing group, e.g., R₂ is an electron withdrawing group, the olefin corresponds to Formula 1-EWG:

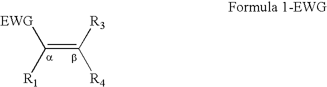

wherein EWG is an electron withdrawing group, R₁ is a substituent of the α-carbon of the ethylenic bond, and R₃ and R₄ are substituents of the β-carbon of the ethylenic bond. In an embodiment, R₁ is hydrogen, hydrocarbyl, substituted hydrocarbyl, or heterocyclo, and R₃ and R₄ are independently hydrogen, hydrocarbyl, substituted hydrocarbyl, heterocyclo, or an electron withdrawing group that is the same as or different from EWG. In one embodiment, R₁ is hydrogen. In another embodiment, R₁ is alkyl or substituted alkyl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, at least one of R₁, R₃ and R₄ is hydrogen and the other two are alkyl or substituted alkyl. In another embodiment, at least two of R₁, R₃, and R₄ are hydrogen and the other is alkyl or substituted alkyl. In another embodiment, R₁, R₃, and R₄ are all hydrogen. In one embodiment, R₃, R₄ and the β-carbon form a carbocyclic or heterocyclic ring. In another embodiment, R₁, R₃, the α-carbon, and the β-carbon form a carbocyclic or heterocyclic ring. In another embodiment, R₁, R₄, the α-carbon, and the β-carbon form a carbocyclic or heterocyclic ring.

When the olefin corresponds to Formula 1-EWG and one of R₄ and R₃ is an electron withdrawing group, the olefin corresponds to Formula 1-EWG-trans or Formula 1-EWG-cis, respectively:

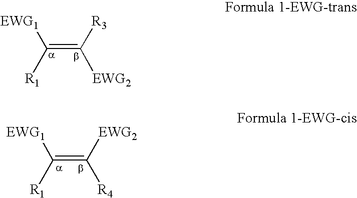

wherein EWG₁ and EWG₂ are electron withdrawing groups and are the same or are different, wherein R₁ is a substituent of the α-carbon of the ethylenic bond, and wherein R₃ and R₄ are substituents of the β-carbon of the ethylenic bond. In this embodiment, R₁, R₃ and R₄ are preferably independently hydrogen, hydrocarbyl, substituted hydrocarbyl or heterocyclo. In one embodiment, R₁ is hydrogen. In another embodiment, R₁ is alkyl or substituted alkyl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment corresponding to Formula 1-EWG-trans, both R₁ and R₃ are hydrogen; in another embodiment corresponding to Formula 1-EWG-cis, both R₁ and R₄ are hydrogen. In one embodiment corresponding to Formula 1-EWG-trans, R₁, R₃, the α-carbon, and the β-carbon form a carbocyclic or heterocyclic ring. In another embodiment corresponding to Formula 1-EWG-cis, R₁, R₄, the α-carbon, and the β-carbon form a carbocyclic or heterocyclic ring.

When the olefin corresponds to Formula 1-EWG, and one of R₁, R₃, and R₄ is hydrogen, the olefin corresponds to Formula 2a-EWG, Formula 2b-EWG, or Formula 2c-EWG:

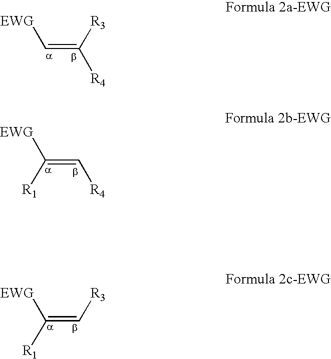

wherein EWG is an electron withdrawing group, R₁ is a substituent of the α-carbon of the ethylenic bond, and R₃ and R₄ are substituents of the β-carbon of the ethylenic bond. In this embodiment, R₁ is preferably independently hydrogen, hydrocarbyl, substituted hydrocarbyl or heterocyclo. In a further embodiment, R₃ and R₄ are preferably independently hydrogen, hydrocarbyl, substituted hydrocarbyl, heterocyclo, or an electron withdrawing group that is the same as or different from EWG. In one embodiment, R₁ is hydrogen. In another embodiment, R₁ is alkyl or substituted alkyl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment corresponding to Formula 2a-EWG, both R₃ and R₄ are hydrogen; in another embodiment corresponding to Formula 2b-EWG, both R₁ and R₄ are hydrogen; in another embodiment corresponding to Formula 2c-EWG, both R₁ and R₃ are hydrogen. In one embodiment corresponding to Formula 2c-EWG, R₁, R₃, the α-carbon, and the β-carbon form a carbocyclic or heterocyclic ring. In another embodiment corresponding to Formula 2b-EWG, R₁, R₄, the α-carbon, and the β-carbon form a carbocyclic or heterocyclic ring. In another embodiment corresponding to Formula 2a-EWG, R₃, R₄, and the β-carbon form a carbocyclic or heterocyclic ring.

In one preferred embodiment, the olefin corresponds to Formula 1-EWG, R₁ is hydrogen, and at least one of R₃ and R₄ is hydrogen. Olefins having this substitution pattern are depicted by Formula 3-EWG:

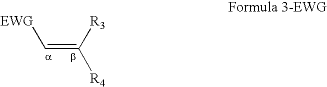

wherein EWG is an electron withdrawing group, and at least one of R₃ and R₄ is hydrogen, while the other is hydrogen, hydrocarbyl, substituted hydrocarbyl, heterocyclo or an electron withdrawing group which is the same as or different from EWG.

When one of R₃ and R₄ is hydrogen and the other is a moiety other than hydrogen, the olefin corresponds to Formula 3-EWG-trans or Formula 3-EWG-cis:

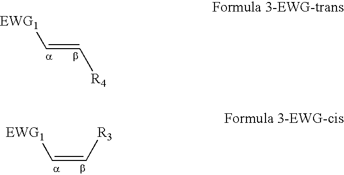

wherein EWG₁ is an electron withdrawing group, R₃ and R₄ are independently hydrogen, hydrocarbyl, substituted hydrocarbyl, heterocyclo, or EWG₂, EWG₂ is an electron withdrawing group, and EWG₁ and EWG₂ are the same or are different. In one embodiment, R₃ is hydrogen. In another embodiment, R₃ is alkyl or substituted alkyl. In another embodiment, R₃ is EWG₂, wherein EWG₁ and EWG₂ can be the same or different. In one embodiment, R₄ is hydrogen. In another embodiment, R₄ is alkyl or substituted alkyl. In another embodiment, R₄ is EWG₂, wherein EWG₁ and EWG₂ can be the same or different.

In another preferred embodiment, the olefin corresponds to Formula 1 and R₁, R₃ and R₄ are hydrogen. Olefins having this substitution pattern are depicted by Formula 4-EWG:

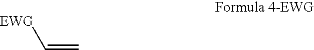

wherein EWG is a substituent of an ethylenic carbon, and EWG is an electron withdrawing group.

In general, the olefin's electron withdrawing group(s), for example, EWG, EWG₁ or EWG₂ as depicted in Formula 1-EWG, Formula 1-EWG-trans, Formula 1-EWG-cis, Formula 2a-EWG, Formula 2b-EWG, Formula 2c-EWG, Formula 3-EWG, Formula 3-EWG-trans, Formula 3-EWG-cis, or Formula 4-EWG, is any substituent that draws electrons away from the ethylenic bond. Exemplary electron withdrawing groups include hydroxy, alkoxy, mercapto, halogens, carbonyls, sulfonyls, nitrile, quaternary amines, nitro, trihalomethyl, imine, amidine, oxime, thioketone, thioester, or thioamide. In one embodiment, the electron withdrawing group(s) is/are hydroxy, alkoxy, mercapto, halogen, carbonyl, sulfonyl, nitrile, quaternary amine, nitro, or trihalomethyl. In another embodiment, the electron withdrawing group(s) is/are halogen, carbonyl, nitrile, quaternary amine, nitro, or trihalomethyl. In another embodiment, the electron withdrawing group(s) is/are halogen, carbonyl, nitrile, nitro, or trihalomethyl. When the electron withdrawing group is alkoxy, it generally corresponds to the formula —OR where R is hydrocarbyl, substituted hydrocarbyl, or heterocyclo. When the electron withdrawing group is mercapto, it generally corresponds to the formula —SR where R is hydrogen, hydrocarbyl, substituted hydrocarbyl or heterocyclo. When the electron withdrawing group is a halogen atom, the electron withdrawing group may be fluoro, chloro, bromo, or iodo; typically, it will be fluoro or chloro. When the electron withdrawing group is a carbonyl, it may be an aldehyde (—C(O)H), ketone (—C(O)R), ester (—C(O)OR), acid (—C(O)OH), acid halide (—C(O)X), amide (—C(O)NRₐR_{b}), or anhydride (—C(O)OC(O)R) where R is hydrocarbyl, substituted hydrocarbyl or heterocyclo, Rₐ and R_{b} are independently hydrogen, hydrocarbyl, substituted hydrocarbyl or heterocyclo, and X is a halogen atom. When the electron withdrawing group is a sulfonyl, it may be an acid (—SO₃H) or a derivative thereof (—SO₂R) where R is hydrocarbyl, substituted hydrocarbyl or heterocyclo. When the electron withdrawing group is a quaternary amine, it generally corresponds to the formula —N⁺RₐR_{b}R_{c} where Rₐ, R_{b} and R_{c} are independently hydrogen, hydrocarbyl, substituted hydrocarbyl or heterocyclo. When the electron withdrawing group is a trihalomethyl, it is preferably trifluoromethyl or trichloromethyl. In each of the foregoing exemplary electron withdrawing groups containing the variable “X”, in one embodiment, X may be chloro or fluoro, preferably fluoro. In each of the foregoing exemplary electron withdrawing groups containing the variable “R”, R may be alkyl. In each of the foregoing exemplary electron withdrawing groups containing the variable “Rₐ” and “R_{b}”, Rₐ and R_{b} may independently be hydrogen or alkyl.

In an embodiment, α,β-unsaturated carbonyl compounds and α,β-unsaturated nitriles are cyclopropanated. In one embodiment, therefore, the olefin's electron withdrawing group(s), for example, EWG, EWG₁ or EWG₂ as depicted in Formula 1-EWG, Formula 1-EWG-trans, Formula 1-EWG-cis, Formula 2a-EWG, Formula 2b-EWG, Formula 2c-EWG, Formula 3-EWG, Formula 3-EWG-trans, Formula 3-EWG-cis, or Formula 4-EWG, is/are a carbonyl or a nitrile. For other applications, it may nonetheless be preferred that one or both of the ethylenic carbons of the olefin possess a quaternary amine, nitro, or trihalomethyl substituent.

In accordance with one preferred embodiment, the electron withdrawing group(s) is/are a halide, aldehyde, ketone, ester, carboxylic acid, amide, acyl chloride, trifluoromethyl, nitrile, sulfonic acid, ammonia, amine, or a nitro group. In this embodiment, the electron withdrawing group(s) correspond to one of the following chemical structures: —X, —C(O)H, —C(O)R, —C(O)OR, —C(O)OH, —C(O)X, —C(X)₃, —CN, —SO₃H, —N⁺H₃, —N⁺R₃, or —N⁺O₂ where R is hydrocarbyl, substituted hydrocarbyl or heterocyclo and X is halogen. Exemplary halogens include fluorine, chlorine, bromine, and iodine. Particularly preferred halogens are fluorine and chlorine.

### Diazosulfones

In general, the carbene source used to cyclopropanate the olefin is a diazosulfone, selected from the group consisting of aromatic diazosulfones and non-aromatic diazosulfones. In one preferred embodiment, the diazosulfone corresponds to the following Formula 6:

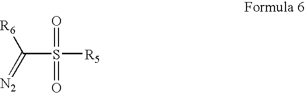

wherein R₅ and R₆ are independently hydrogen, hydrocarbyl, substituted hydrocarbyl, or heterocyclo. In one embodiment, R₅ and R₆ are independently hydrogen, hydrocarbyl or substituted hydrocarbyl. In one embodiment, R₆ is hydrogen. In one embodiment, R₆ is hydrogen and R₅ is hydrocarbyl or substituted hydrocarbyl; for example, in this embodiment, R₆ is hydrogen and R₅ is alkyl, alkenyl, alkynyl, phenyl, alkyl or heterosubstituted phenyl. In one preferred embodiment, R₆ is hydrogen and R₅ is C₁₋₈ alkyl, phenyl, C₁₋₈ alkyl substituted phenyl, substituted phenyl, alkyl or heterosubstituted phenyl. In another embodiment, R₆ is hydrocarbyl or substituted hydrocarbyl; for example, in this embodiment, R₆ is hydrocarbyl or substituted hydrocarbyl and R₅ is alkyl, alkenyl, alkynyl, phenyl, alkyl or heterosubstituted phenyl. In another embodiment, R₆ is alkyl or substituted alkyl. In one embodiment, R₅ is hydrogen. In one embodiment, R₅ is hydrogen, alkyl, alkenyl, alkynyl, phenyl, or aryl. In another embodiment, R₅ is substituted alkyl, substituted alkenyl, substituted alkynyl, substituted phenyl, or heterosubstituted phenyl. In a preferred embodiment, R₅ is optionally substituted phenyl, including but not limited to toluoyl, nitrophenyl, or methoxyphenyl. In a particularly preferred embodiment, R₆ is hydrogen and R₅ is phenyl, p-toluoyl, p-nitrophenyl, or p-methoxyphenyl.

### Cyclopropanes

In general, the cyclopropanes of the present invention correspond to the following formula:

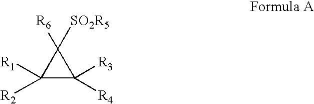

wherein R₁, R₂, R₃, R₄, are independently hydrogen, hydrocarbyl, substituted hydrocarbyl, heterocyclo, or an electron-withdrawing group, and R₅ and R₆ are independently hydrogen, hydrocarbyl, substituted hydrocarbyl, or heterocyclo. In one embodiment, the cyclopropane corresponds to Formula A and R₁, R₂, R₃, R₄, R₅, and R₆ are independently hydrocarbyl or substituted hydrocarbyl. In one embodiment, at least one of R₁ and R₂, and at least one of R₃ and R₄, are hydrogen. Thus, for example, when the cyclopropane is derived from an α,β-unsaturated alkene, R₁ and R₂, or R₃ and R₄ will be hydrogen and at least one of R₁, R₂, R₃ and R₄ will be optionally substituted alkyl, optionally substituted alkenyl, optionally substituted alkynyl, or optionally substituted aryl. In one preferred embodiment, three of R₁, R₂, R₃ and R₄ are hydrogen and the other is optionally substituted alkyl, optionally substituted alkenyl, optionally substituted alkynyl, optionally substituted aryl. Thus, for example, in one embodiment, R₁, R₃ and R₄ are hydrogen and R₂ is alkyl, aryl, substituted phenyl, —CN, —C(O)R₂₂, or —C(O)OR₂₂ wherein R₂₂ is optionally substituted alkyl, optionally substituted alkenyl, optionally substituted optionally substituted alkynyl, or optionally substituted aryl.

Still referring to Formula A, in one preferred embodiment, three of R₁, R₂, R₃ and R₄ are hydrogen and the other is optionally substituted alkyl, optionally substituted alkenyl, optionally substituted alkynyl, or optionally substituted aryl. Thus, for example, in one embodiment, R₁, R₃ and R₄ are hydrogen and R₂ is optionally substituted phenyl, —CN, —C(O)R₂₂, or —C(O)OR₂₂ wherein R₂₂ is optionally substituted alkyl, optionally substituted alkenyl, optionally substituted optionally substituted alkynyl, or optionally substituted aryl. In another example in which three of R₁, R₃ and R₄ are hydrogen: R₂ is preferably —CN, —C(O)CH₃, —C(O)OCH₃, or —C(O)OC₂H₅; more preferably R₂ is phenyl, tert-butyl phenyl, methoxyphenyl, trifluoromethyl phenyl, nitrophenyl, or naphthyl; even more preferably, R₂ is phenyl, p-tert-butyl phenyl, p-methoxyphenyl, p-trifluoromethyl phenyl, 3-nitrophenyl, or naphthyl.

Still referring to Formula A, in one embodiment, R₆ is hydrogen. In another embodiment, R₆ is alkyl or substituted alkyl. In one embodiment, R₅ is hydrogen. In one embodiment, R₅ is hydrogen, alkyl, alkenyl, alkynyl, phenyl, or aryl. In another embodiment, R₅ is substituted alkyl, substituted alkenyl, substituted alkynyl, substituted phenyl, substituted aryl, or heterosubstituted phenyl. In a preferred embodiment, R₅ is phenyl, methylphenyl, nitrophenyl, or methoxyphenyl. In a particularly preferred embodiment, R₆ is hydrogen and R₅ is phenyl, p-methylphenyl, p-nitrophenyl, or p-methoxyphenyl.

In one embodiment, the cyclopropane has the following structure

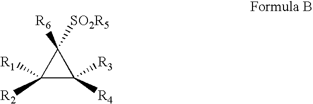

wherein R₁, R₂, R₃, R₄, R₅, and R₆ are as previously described in connection with each embodiment of Formula A. In one embodiment, the cyclopropane corresponds to Formula B and R₁, R₂, R₃, R₄, are independently hydrogen, hydrocarbyl, substituted hydrocarbyl, heterocyclo, or an electron-withdrawing group, and R₅ and R₆ are independently hydrogen, hydrocarbyl, substituted hydrocarbyl, or heterocyclo. In one embodiment, at least one of R₁ and R₂, and at least one of R₃ and R₄, are hydrogen. In an embodiment wherein the cyclopropane is derived from an α,β-unsaturated olefin, R₁ or R₂, and R₃ or R₄, will be hydrogen and at least one of R₁, R₂, R₃ and R₄ will be optionally substituted alkyl, optionally substituted alkenyl, optionally substituted alkynyl, or optionally substituted aryl. In an embodiment wherein the cyclopropane is derived from a terminal olefin, R₁ and R₂, or R₃ and R₄, will be hydrogen, and at least one of R₁, R₂, R₃ and R₄ will be optionally substituted alkyl, optionally substituted alkenyl, optionally substituted alkynyl, or optionally substituted aryl.

Still referring to Formula B, in one preferred embodiment, three of R₁, R₂, R₃ and R₄ are hydrogen and the other is optionally substituted alkyl, optionally substituted alkenyl, optionally substituted alkynyl, or optionally substituted aryl. Thus, for example, in one embodiment, R₁, R₃ and R₄ are hydrogen and R₂ is optionally substituted phenyl, —CN, —C(O)R₂₂, or —C(O)OR₂₂ wherein R₂₂ is optionally substituted alkyl, optionally substituted alkenyl, optionally substituted optionally substituted alkynyl, or optionally substituted aryl. In another example in which three of R₁, R₃ and R₄ are hydrogen: R₂ is preferably —CN, —C(O)CH₃, —C(O)OCH₃, or —C(O)OC₂H₅; more preferably R₂ is phenyl, tert-butyl phenyl, methoxyphenyl, trifluoromethyl phenyl, nitrophenyl, or naphthyl; even more preferably, R₂ is phenyl, p-tert-butyl phenyl, p-methoxyphenyl, p-trifluoromethyl phenyl, 3-nitrophenyl, or naphthyl.

Still referring to Formula B, in one embodiment, R₆ is hydrogen. In another embodiment, R₆ is alkyl or substituted alkyl. In one embodiment, R₅ is hydrogen. In one embodiment, R₅ is hydrogen, alkyl, alkenyl, alkynyl, phenyl, or aryl. In another embodiment, R₅ is substituted alkyl, substituted alkenyl, substituted alkynyl, substituted phenyl, substituted aryl, or heterosubstituted phenyl. In a preferred embodiment, R₅ is phenyl, methylphenyl, nitrophenyl, or methoxyphenyl. In a particularly preferred embodiment, R₆ is hydrogen and R₅ is phenyl, p-methylphenyl, p-nitrophenyl, or p-methoxyphenyl.

In a preferred embodiment, when the cyclopropane corresponds to Formula A and when R₂, R₃, R₄, and R₆ are hydrogen, the cyclopropane corresponds to the following structure

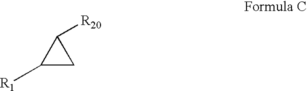

wherein R₁ is hydrogen, hydrocarbyl, substituted hydrocarbyl, heterocyclo, or an electron withdrawing group, and R₂₀ is a sulfonyl group corresponding to SO₂R₂₄ wherein R₂₄ is hydrogen, hydrocarbyl, substituted hydrocarbyl, or heterocyclo. In an embodiment, R₁ is alkyl or substituted alkyl. In a preferred embodiment, R₁ is optionally substituted phenyl. In a further preferred embodiment, R₁ is phenyl, tert-butylphenyl, methoxyphenyl, trifluorophenyl, or nitrophenyl. In an even further preferred embodiment, R₁ is phenyl, 4-tert-butylphenyl, 4-methoxyphenyl, 4-trifluorophenyl, or 3-nitrophenyl. In another embodiment, R₁ is naphthyl. In another embodiment, R₁ is an electron withdrawing group. In another preferred embodiment, R₁ is —CN, —C(O)CH₃, —C(O)OCH₃, or —C(O)OC₂H₅. In one preferred embodiment, R₂₀ is an optionally substituted phenyl sulfonyl; in this preferred embodiment, R₂₄ is an optionally substituted phenyl, including without limitation phenyl, toluene, methoxyphenyl, or nitrophenyl. In another preferred embodiment, R₂₀ is tosyl, methoxyphenylsulfonyl, or nitrophenylsulfonyl. In another more preferred embodiment, R₂₀ is tosyl, 4-methoxyphenylsulfonyl, or 4-nitrophenylsulfonyl.

As illustrated more fully in the examples, the diastero- and enantio-selectivity can be influenced, at least in part, by selection of the metal porphyrin complex. Similarly, stereoselectivity of the reaction may also be influenced by the selection of chiral porphyrin ligands with desired electronic, steric, and chiral environments. Accordingly, the catalytic system of the present invention may advantageously be used to control stereoselectivity.

### Metal Porphyrin Complexes

An aspect of the present invention is a process for the cyclopropanation of olefins in the presence of a catalyst. In an embodiment, the catalyst is a metal porphyrin complex. In one embodiment, the metal of the metal porphyrin complex is a transition metal. Thus, for example, the metal, M, may be any of the 30 metals in the 3d, 4d, and 5d transition metal series of the Periodic Table of the Elements, including the 3d series that includes Sc, Ti, V, Cr, Mn, Fe, Co, Ni, Cu, and Zn; the 4d series that includes Y, Zr, Nb, Mo, Tc, Ru, Rh, Pd, Ag and Cd; and the 5d series that includes Lu, Hf, Ta, W, Re, Os, Ir, Pt, Au and Hg. In some embodiments, M is a transition metal from the 3d series. In some embodiments, M is selected from the group consisting of Co, Zn, Fe, Ru, Mn, and Ni. In some embodiments, M is selected from the group consisting of Co, Fe, and Ru. In some embodiments, M is Co.

The porphyrin with which the metal is complexed may be any of a wide range of porphyrins known in the art. Exemplary porphyrins are described in U.S. Patent Publication Nos. 2005/0124596 and 2006/0030718 and U.S. Pat. No. 6,951,935 (each of which is incorporated herein by reference, in its entirety). Exemplary porphyrins are also described in Chen et al., Bromoporphyrins as Versatile Synthons for Modular Construction of Chiral Porphyrins: Cobalt-Catalyzed Highly Enantioselective and Diastereoselective Cyclopropanation (J. Am. Chem. Soc. 2004), which is incorporated herein by reference in its entirety.

In a preferred embodiment, the porphyrin is complexed with cobalt. The porphyrin with which cobalt is complexed may be any of a wide range of porphyrins known in the art. Exemplary porphyrins are described in U.S. Patent Publication Nos. 2005/0124596 and 2006/0030718 and U.S. Pat. No. 6,951,935 (each of which is incorporated herein by reference, in its entirety). Exemplary porphyrins are also described in Chen et al., Bromoporphyrins as Versatile Synthons for Modular Construction of Chiral Porphyrins: Cobalt-Catalyzed Highly Enantioselective and Diastereoselective Cyclopropanation (J. Am. Chem. Soc. 2004), which is incorporated herein by reference in its entirety.

In one embodiment of the present invention, the olefin is treated with a diazosulfone in the presence of a cobalt complex. In one embodiment, the cobalt complex is a cobalt (II) complex. In a preferred embodiment, the cobalt (II) complex is a cobalt porphyrin complex. In one embodiment, the cobalt porphyrin complex is a cobalt (II) porphyrin complex. In one particularly preferred embodiment, the cobalt porphyrin complex is a D₂-symmetric chiral porphyrin complex corresponding to the following structure:

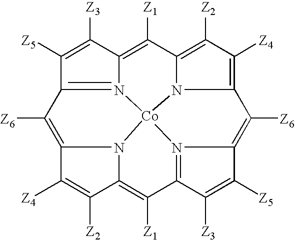

wherein each Z₁, Z₂, Z₃, Z₄, Z₅ and Z₆ are each independently selected from the group consisting of X, H, alkyl, substituted alkyls, arylalkyls, aryls and substituted aryls; and X is selected from the group consisting of halogen, trifluoromethanesulfonate (OTf), haloaryl and haloalkyl. In a preferred embodiment, Z₂, Z₃, Z₄ and Z₅ are hydrogen, Z₁ is a substituted phenyl, and Z₆ is substituted phenyl, and Z₁ and Z₆ are different. In one particularly preferred embodiment, Z₂, Z₃, Z₄ and Z₅ are hydrogen, Z₁ is substituted phenyl, and Z₆ is substituted phenyl and Z₁ and Z₆ are different and the porphyrin is a chiral porphyrin. In one even further preferred embodiment, Z₂, Z₃, Z₄ and Z₅ are hydrogen, Z₁ is substituted phenyl, and Z₆ is substituted phenyl and Z₁ and Z₆ are different and the porphyrin has D₂-symmetry.

Exemplary cobalt porphyrins include the following:

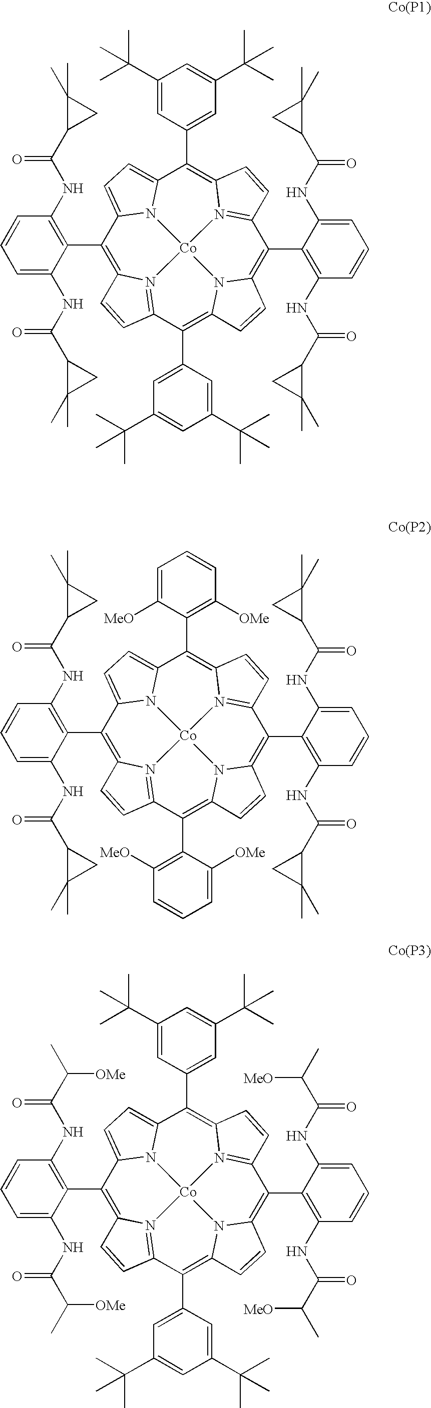

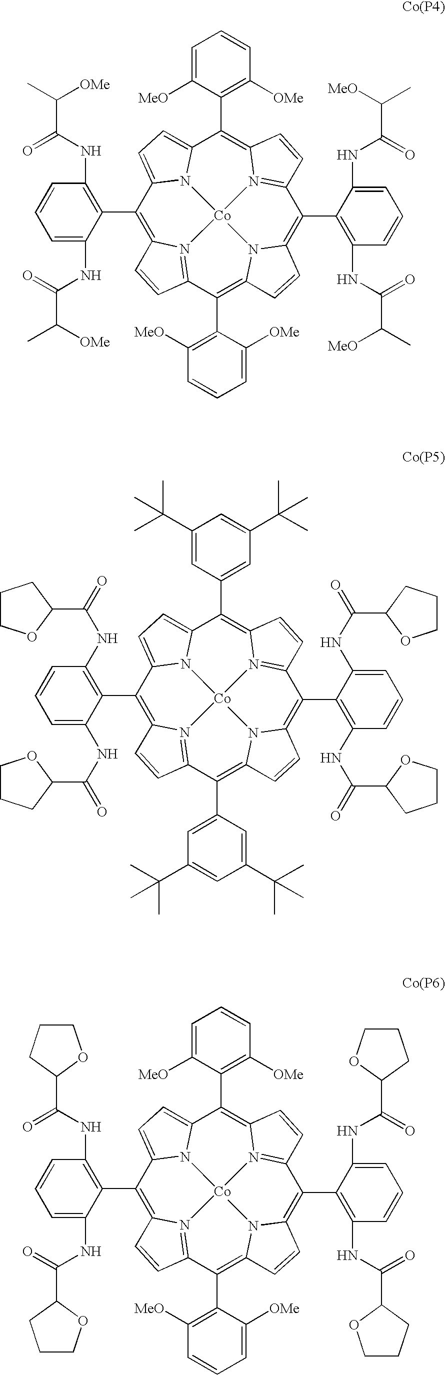

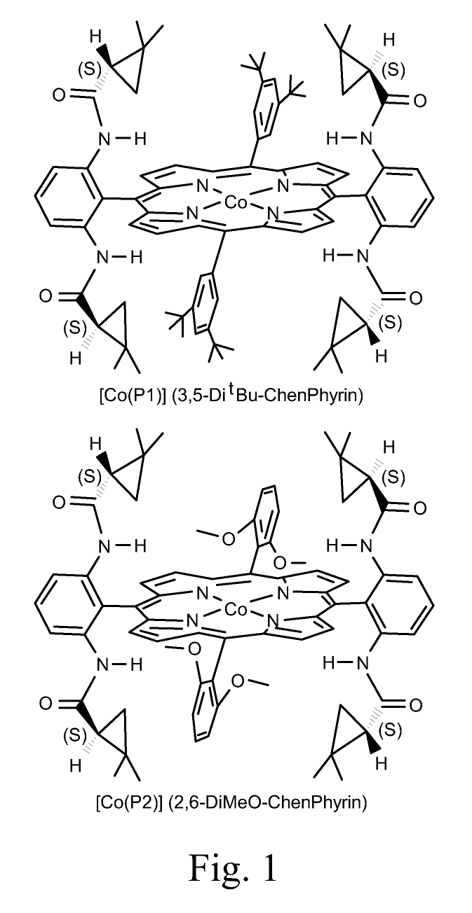

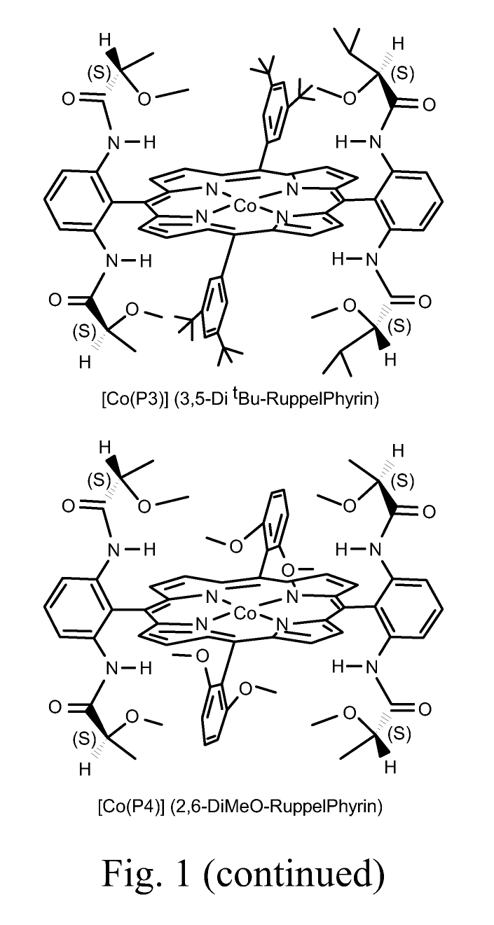

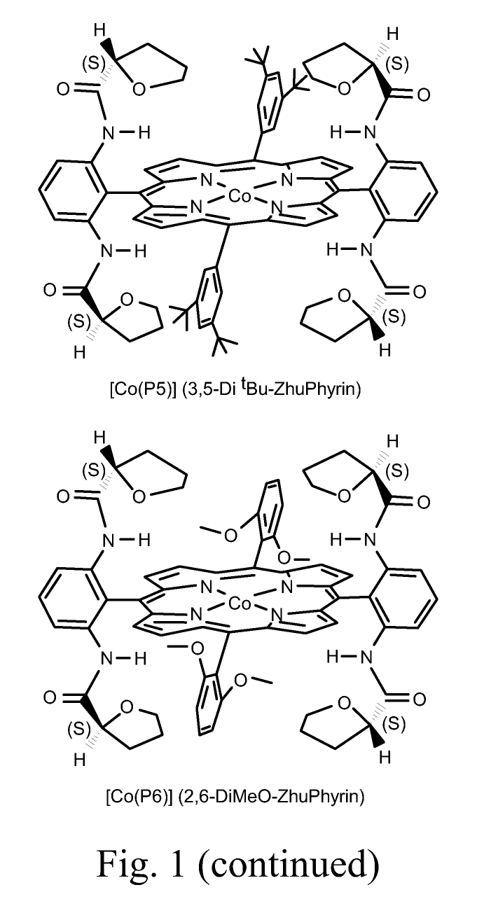

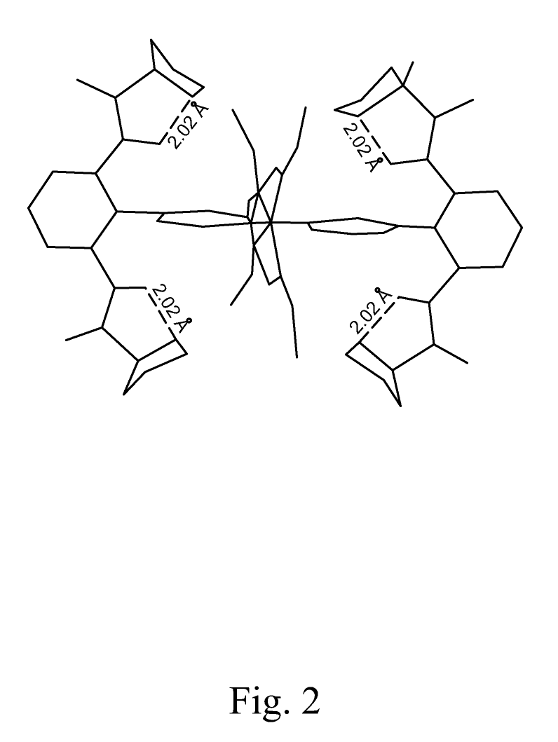

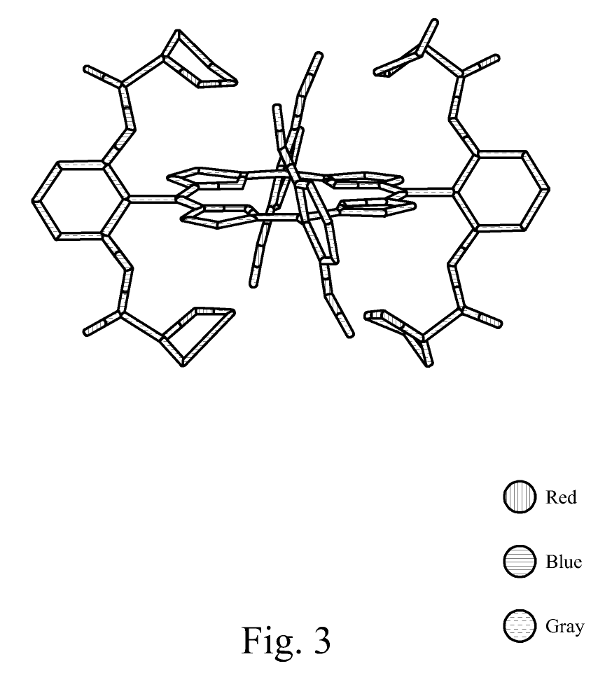

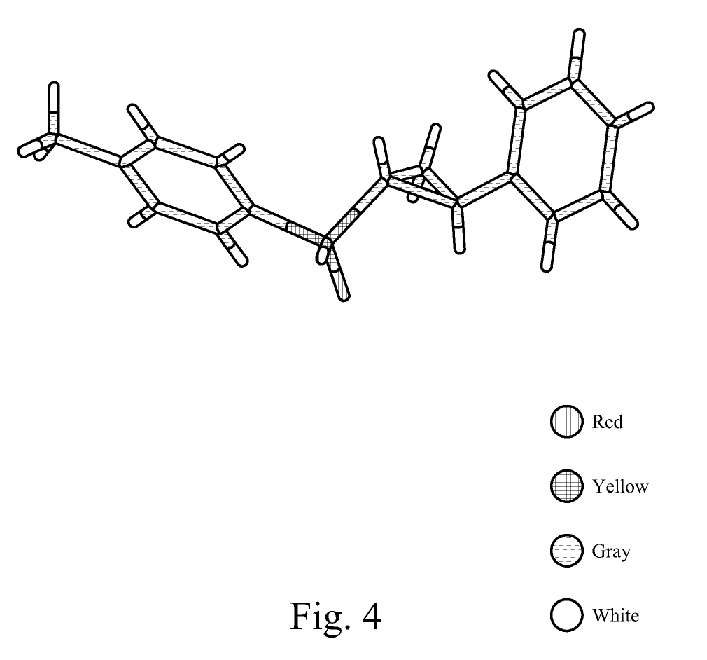

The stereochemistry of these exemplary cobalt porphyrin complexes is shown in FIG. 1.

### Cyclopropanation Reactions

In one embodiment, the cyclopropanation reaction is as depicted in Reaction Scheme I:

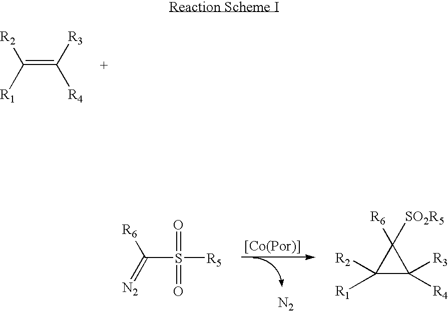

wherein R₁, R₂, R₃, R₄, R₅, and R₆ are as previously described in connection with the alkene and diazosulfone and Co(Por) is a cobalt porphyrin complex. In one embodiment, the cyclopropanation reaction is as depicted in Reaction Scheme I wherein R₁, R₂, R₃, and R₄ are independently hydrogen, hydrocarbyl, substituted hydrocarbyl, or heterocyclo; wherein R₅ and R₆ are independently hydrogen, hydrocarbyl, substituted hydrocarbyl, or heterocyclo; and wherein Co(Por) is a cobalt porphyrin complex. In one embodiment, R₁ is hydrogen. In another embodiment, R₁ is alkyl or substituted alkyl. In one embodiment, R₂ is hydrogen. In another embodiment, R₂ is alkyl or substituted alkyl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, two of R₁, R₂, R₃ and R₄ are hydrogen. In another embodiment, three of R₁, R₂, R₃, and R₄ are hydrogen. In one embodiment, R₁, R₂ and the α-carbon, or R₃, R₄ and the β-carbon form a carbocyclic or heterocyclic ring. In another embodiment, R₁, R₃, the α-carbon, and the β-carbon, or R₂, R₄, the α-carbon, and the (3-carbon form a carbocyclic or heterocyclic ring. In another embodiment, R₁, R₄, the α-carbon, and the β-carbon or R₂, R₃, the α-carbon, and the (3-carbon form a carbocyclic or heterocyclic ring. In one preferred embodiment, at least one of R₁, R₂, R₃, and R₄ is alkyl, aryl, substituted phenyl, —CN, —C(O)R₂₂, or —C(O)OR₂₂ wherein R₂₂ is hydrogen, optionally substituted alkyl, optionally substituted alkenyl, optionally substituted alkynyl, or optionally substituted aryl. In a further preferred embodiment, at least one of R₁, R₂, R₃ and R₄ is —CN, —C(O)CH₃, —C(O)OCH₃, or —C(O)OC₂H₅. In another preferred embodiment, at least one of R₁, R₂, R₃ and R₄ is phenyl, tert-butyl phenyl, methoxyphenyl, trifluoromethyl phenyl, nitrophenyl, or naphthyl. In a further preferred embodiment, at least one of R₁, R₂, R₃ and R₄ is phenyl, p-tert-butyl phenyl, p-methoxyphenyl, p-trifluoromethyl phenyl, 3-nitrophenyl, or naphthyl. In preferred embodiments, the olefin is an aromatic olefin, an α,β-unsaturated ester, an α,β-unsaturated ketone, or an α,β-unsaturated nitrile. In one embodiment, R₆ is hydrogen. In another embodiment, R₆ is alkyl or substituted alkyl. In one embodiment, R₅ is hydrogen. In one embodiment, R₅ is hydrogen, alkyl, alkenyl, alkynyl, phenyl, or aryl. In another embodiment, R₅ is substituted alkyl, substituted alkenyl, substituted alkynyl, substituted phenyl, substituted aryl, or heterosubstituted phenyl. In a preferred embodiment, R₅ is optionally substituted phenyl, including but not limited to phenyl, methylphenyl, nitrophenyl, or methoxyphenyl. In a particularly preferred embodiment, R₆ is hydrogen and R₅ is phenyl, p-methylphenyl, p-nitrophenyl, or p-methoxyphenyl.

In an embodiment, the cyclopropanation reaction is as depicted in Reaction Scheme II:

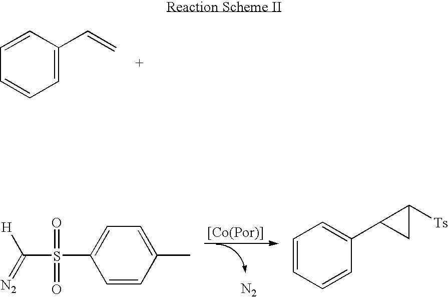

wherein Ts is a tosyl group and Co(Por) is a cobalt porphyrin complex.

In another embodiment, the cyclopropanation reaction is as depicted in Reaction Scheme III:

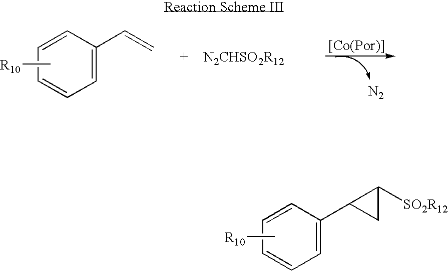

wherein R₁₀ is hydrogen, hydrocarbyl, substituted hydrocarbyl, or heterocyclo, wherein R₁₂ is hydrogen, hydrocarbyl, substituted hydrocarbyl, or heterocyclo, and wherein Co(Por) is a cobalt porphyrin complex. In a preferred embodiment, R₁₀ is t-butyl, —OCH₃, —CF₃, or —NO₂. In a preferred embodiment, R₁₂ is hydrogen, alkyl, alkenyl, alkynyl, phenyl, or aryl. In another preferred embodiment, R₁₂ is substituted alkyl, substituted alkenyl, substituted alkynyl, substituted phenyl, substituted aryl, or heterosubstituted phenyl. In yet another preferred embodiment, R₁₂ is optionally substituted phenyl, including but not limited to phenyl, methylphenyl, nitrophenyl, or methoxyphenyl. In one preferred embodiment, R₁₂ is phenyl, p-methylphenyl, p-nitrophenyl, or p-methoxyphenyl.

In another embodiment, the cyclopropanation reaction is as depicted in Reaction Scheme IV:

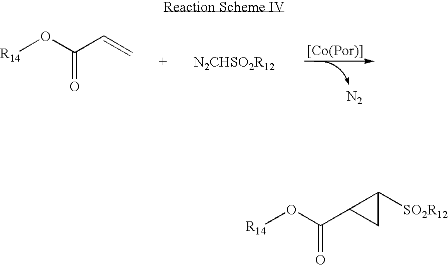

wherein R₁₄ is hydrogen, hydrocarbyl, substituted hydrocarbyl or heterocyclo, wherein R₁₂ is hydrogen, hydrocarbyl, substituted hydrocarbyl, or heterocyclo, and wherein Co(Por) is a cobalt porphyrin complex. In a preferred embodiment, R₁₄ is alkyl or substituted alkyl. In another preferred embodiment, R₁₄ is methyl or ethyl. In a preferred embodiment, R₁₂ is hydrogen, alkyl, alkenyl, alkynyl, phenyl, or aryl. In a more preferred embodiment, R₁₂ is substituted alkyl, substituted alkenyl, substituted alkynyl, substituted phenyl, substituted aryl, or heterosubstituted phenyl. In yet another preferred embodiment, R₁₂ is optionally substituted phenyl, including but not limited to phenyl, methylphenyl, nitrophenyl, or methoxyphenyl. In one preferred embodiment, R₁₂ is phenyl, p-methylphenyl, p-nitrophenyl, or p-methoxyphenyl.

In another embodiment, the cyclopropanation reaction is as depicted in Reaction Scheme V:

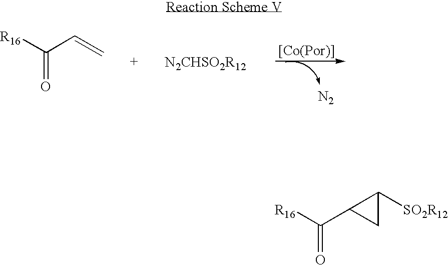

wherein R₁₆ is hydrogen, hydrocarbyl, or substituted hydrocarbyl, wherein R₁₂ is hydrogen, hydrocarbyl, substituted hydrocarbyl, or heterocyclo, and wherein Co(Por) is a cobalt porphyrin complex. In an embodiment, R₁₆ is alkyl or substituted alkyl. In a preferred embodiment, R₁₆ is methyl. In a preferred embodiment, R₁₂ is hydrogen, alkyl, alkenyl, alkynyl, phenyl, or aryl. In a more preferred embodiment, R₁₂ is substituted alkyl, substituted alkenyl, substituted alkynyl, substituted phenyl, substituted aryl, or heterosubstituted phenyl. In yet another preferred embodiment, R₁₂ is optionally substituted phenyl, including but not limited to phenyl, methylphenyl, nitrophenyl, or methoxyphenyl. In one preferred embodiment, R₁₂ is phenyl, p-methylphenyl, p-nitrophenyl, or p-methoxyphenyl.

In another embodiment, the cyclopropanation reaction is as depicted in Reaction Scheme VI:

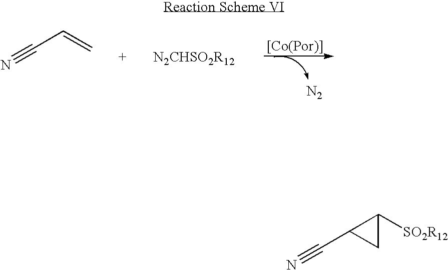

wherein R₁₂ is hydrogen, hydrocarbyl, substituted hydrocarbyl, or heterocyclo, and wherein Co(Por) is a cobalt porphyrin complex. In a preferred embodiment, R₁₂ is hydrogen, alkyl, alkenyl, alkynyl, phenyl, or aryl. In another preferred embodiment, R₁₂ is substituted alkyl, substituted alkenyl, substituted alkynyl, substituted phenyl, substituted aryl, or heterosubstituted phenyl. In yet another preferred embodiment, R₁₂ is optionally substituted phenyl, including but not limited to phenyl, methylphenyl, nitrophenyl, or methoxyphenyl. In one preferred embodiment, R₁₂ is phenyl, p-methylphenyl, p-nitrophenyl, or p-methoxyphenyl.

In accordance with one embodiment of the present invention, an olefin is converted to a cyclopropane as illustrated in Reaction Scheme 1:

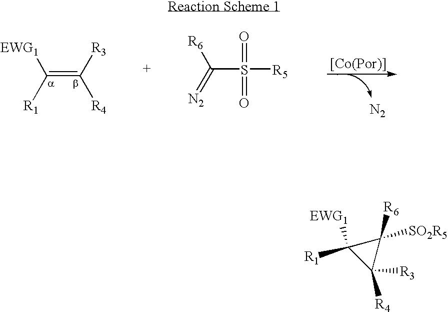

wherein Co(Por) is a cobalt porphyrin complex, R₁ is hydrogen, hydrocarbyl, substituted hydrocarbyl, or heterocyclo, R₃ and R₄ are independently hydrogen, hydrocarbyl, substituted hydrocarbyl, heterocyclo, or EWG₂, R₅ and R₆ are independently hydrogen, hydrocarbyl, substituted hydrocarbyl, or heterocyclo, and EWG₁ and EWG₂ are each an electron-withdrawing group, and EWG₁ and EWG₂ can be the same or different. In one embodiment, R₆ is hydrogen. In another embodiment, R₆ is alkyl or substituted alkyl. In one embodiment, R₅ is hydrogen. In one embodiment, R₅ is hydrogen, alkyl, alkenyl, alkynyl, phenyl, or aryl. In another embodiment, R₅ is substituted alkyl, substituted alkenyl, substituted alkynyl, substituted phenyl, substituted aryl, or heterosubstituted phenyl. In a preferred embodiment, R₅ is optionally substituted phenyl, including but not limited to phenyl, methylphenyl, nitrophenyl, or methoxyphenyl. In one particularly preferred embodiment, R₆ is hydrogen and R₅ is phenyl, p-methylphenyl, p-nitrophenyl, or p-methoxyphenyl.

In accordance with one embodiment, each of the ethylenic carbons possesses an electron withdrawing group and the cyclopropanation reaction proceeds as depicted in Reaction Scheme 2 or 3:

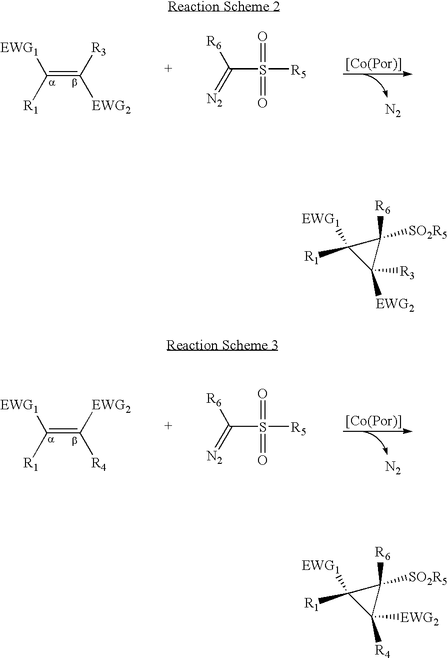

wherein [Co(Por)] is a cobalt porphyrin complex, R₁, R₃, and R₄ are independently hydrogen, hydrocarbyl, substituted hydrocarbyl, or heterocyclo, EWG₁ and EWG₂ are independently an electron-withdrawing group and EWG₁ and EWG₂ can be the same or different, and R₅ and R₆ are independently hydrogen, hydrocarbyl, substituted hydrocarbyl, or heterocyclo. In one embodiment, R₁ is hydrogen. In another embodiment, R₁ is alkyl or substituted alkyl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment corresponding to Reaction Scheme 2, both R₁ and R₃ are hydrogen; in another embodiment corresponding to Reaction Scheme 3, both R₁ and R₄ are hydrogen. In one embodiment corresponding to Reaction Scheme 2, R₁, R₃, the α-carbon, and the (3-carbon form a carbocyclic or heterocyclic ring. In another embodiment corresponding to Reaction Scheme 3, R₁, R₄, the α-carbon, and the β-carbon form a carbocyclic or heterocyclic ring. In one embodiment, R₆ is hydrogen. In another embodiment, R₆ is alkyl or substituted alkyl. In one embodiment, R₅ is hydrogen. In one embodiment, R₅ is hydrogen, alkyl, alkenyl, alkynyl, phenyl, or aryl. In another embodiment, R₅ is substituted alkyl, substituted alkenyl, substituted alkynyl, substituted phenyl, substituted aryl, or heterosubstituted phenyl. In a preferred embodiment, R₅ is optionally substituted phenyl, including but not limited to phenyl, methylphenyl, nitrophenyl, or methoxyphenyl. In one particularly preferred embodiment, R₆ is hydrogen and R₅ is phenyl, p-methylphenyl, p-nitrophenyl, or p-methoxyphenyl.

Under the conditions optimized for asymmetric cyclopropanation with diazoacetates, which required a substoichiometric amount of DMAP due to a positive trans effect, our initial attempts to apply [Co(P1)] as a catalyst to cyclopropanate styrene with tosyldiazomethane met with surprising disappointment (Table 1, entry 1). (Chen et al., Synthesis 2006, 1697). Concurring with the assumption of a competitive carbene transfer to DMAP, removal of DMAP resulted in a dramatic increase of the cyclopropane formation but still exhibited poor enantioselectivity (Table 1, entry 2). Employment of a bulkier ligand P2 bearing meso-2,6-dimethoxyphenyl groups improved the enantioselectivity substantially (Table 1, entry 3). Alteration of the chiral units with acyclic amides but possessing intramolecular O^{...}H—N hydrogen bonding interactions provided chiral porphyrins P3 and P4, Co complexes of which [Co(P3)] and [Co(P4)] gave better results than the respective [Co(P1)] and [Co(P2)] (Table 1, entries 2-5). (For select examples of chiral ligands with Intramolecular hydrogen bonding interactions, see: Morice et al., J. Chem. Soc., Dalton Trans. 1998, 4165; Boitrel et al., Eur. J. Org. Chem. 2001, 4213; Liu et al., J. Am. Chem. Soc. 2006, 128, 14212.) To create an even more rigid and polar chiral environment, the combined incorporation of intramolecular O^{...}H—N hydrogen bonding interactions and cyclic structures led to the design and synthesis of chiral porphyrins P5 and P6 through the use of (S)-(−)-2-tetrahydrofurancarboxamide. This design strategy was evidenced by X-ray crystallographic analysis. While [Co(P5)] provided a better enantioselectivity than the respective [Co(P1)] and [Co(P3)] (Table 1, entry 6), [Co(P6)] proved to be the optimal catalyst, furnishing the desired product in 99% yield and 92% ee (Table 1, entry 7). Varying with enantioselectivity, all the catalysts exhibited excellent diastereoselectivity (Table 1, entries 1-7). It was noted that [Co(P5)] and [Co(P6)] gave a sense of asymmetric induction opposite that of the other catalysts, despite having the same (S) absolute configuration (Table 1).

TABLE 1
Asymmetric Cyclopropanation of Styrene with N₂CHTs
Catalyzed by Cobalt (II) Complexes of Different Chiral Porphyrins^{a}

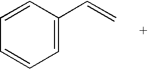

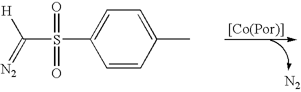

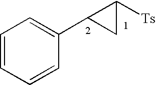

| en- | | | | | | |
|---|---|---|---|---|---|---|
| try | [Co(Por)]^{b} | DMAP^{c} | Yield (%)^{d} | Trans:cis^{e} | Ee (%)^{f} | config^{g} |
| 1 | [Co(P1)] | + | ~6^{h} | >99:01 | 3 | [1R,2S]-(−) |
| 2 | [Co(P1)] | − | 86 | >99:01 | 14 | [1S,2R]-(+) |
| 3 | [Co(P2)] | − | 78 | >99:01 | 56 | [1S,2R]-(+) |
| 4 | [Co(P3)] | − | 60 | >99:0 | 23 | [1S,2R]-(+) |
| 5 | [Co(P4)] | − | 99 | >99:01 | 61 | [1S,2R]-(+) |
| 6 | [Co(P5)] | − | 30 | >99:01 | 54 | [1R,2S]-(−) |
| 7 | [Co(P6)] | − | 99 | >99:01 | 92 | [1R,2S]-(−) |

^{a}Performed in CH₂Cl₂ at room temperature for 24 hours using 1 mol % of [Co(Por)] under N₂ with 1.0 equivalent of styrene and 1.2 equivalent of N₂CHTs; [styrene] = 02.5 M.
^{b}See FIG. 1 and Scheme S1 for structures and syntheses.
^{c}With (+) or without (−) 0.5 equivalent of DMAP.
^{d}Isolated yields.
^{e}Determined by NMR.
^{f}Trans isomer ee was determined by chiral HPLC
^{g}Absolute configuration of major enantiomer determined by X-ray crystal structural analysis and optical rotation.
^{h}Estimated by NMR.

In addition to cyclopropanation of stryene with N₂CHTs, [Co(P6)] was shown to be a general catalyst for a range of aromatic and electron-deficient terminal olefins and with different diazoarylsulfones (Table 2). ([Co(P6)]-based catalytic system was ineffective for multiple substituted olefins and aliphatic olefins.) For example, N₂CHMs and N₂CHNs served equally well as carbene sources as compared to N₂CHTs (Table 2, entries 2-4). Both aromatic olefins with different substituents (Table 2, entries 5-9) and electron-deficient olefins, such as α,β-unsaturated esters (Table 2, entries 10-12), ketones (Table 2, entry 13), and nitriles (Table 2, entry 14), could be effectively cyclopropanated with N₂CHTs by [Co(P6)]. Except for the case of an α,β-unsaturated nitrile (Table 2, entry 14), all the corresponding cyclopropyl sulfones were formed in high enantioselectivity and excellent trans diastereoselectivity (Table 2). Cyclopropyl sulfones that are almost enantiomerically pure (>98% ee) were obtained through a simple recrystallization procedure due to the high crystalline nature of this class of compounds, as exemplified in the styrene and methyl vinyl ketone reactions (Table 2, entries 1 and 13).

TABLE 2
[Co(P6)]-Catalyzed Diastereo- and Enantioselective
Cyclopropanation of Different Alkenes with Various Diazosulfones^{a}

| | | | Y | | | |
|---|---|---|---|---|---|---|
| Entry | Olefin | Cyclopropane | (%)^{b} | T:c^{c} | Ee(%)^{d} | [α]^{e} |
| 1^{f} | 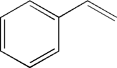 | 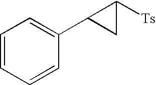 | 99 (66)^{j} | >99:01 (>99:01)^{j} | 92 (>99)^{j} | (−)^{k} |
| 2^{g} | 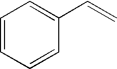 | 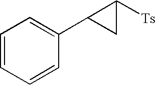 | 81 | >99:01 | 95 | (−)^{k} |
| 3^{g} | 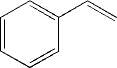 | 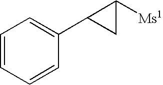 | 97 | >99:01 | 96 | (−) |
| 4^{g} | 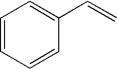 | 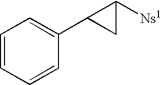 | 99 | >99:01 | 90 | (−) |
| 5^{g} | 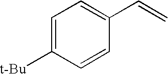 | 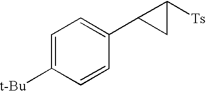 | 57 | >99:01 | 94 | (−) |
| 6^{g} | 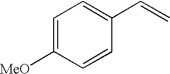 | 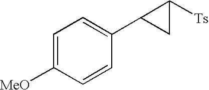 | 72 | >99:01 | 95 | (−) |
| 7^{g} | 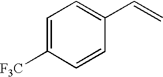 | 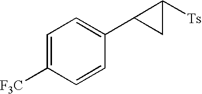 | 88 | >99:01 | 95 | (−) |
| 8^{g} | 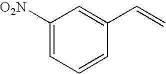 | 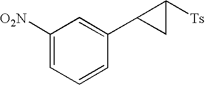 | 77 | >99:01 | 96 | (−) |
| 9^{g} | 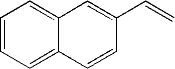 | 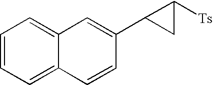 | 81 | >99:01 | 93 | (−) |
| 10^{h} | 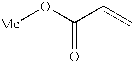 | 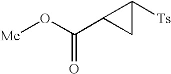 | 96 | 94:06 | 89 | (−) |
| 11ⁱ | 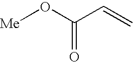 | 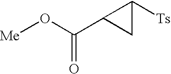 | 64 | >99:01 | 97 | (−) |
| 12^{h} | 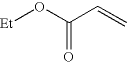 | 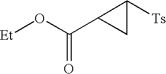 | 72 | >99:01 | 90 | (−) |
| 13^{h} | 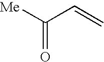 | 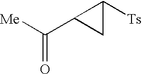 | 93 (81)^{j} | >99:01 (>99:01)^{j} | 89 (98)^{j} | (−) |
| 14^{h} | 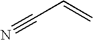 | 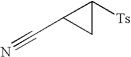 | 81 | 79:21 | 61 | (−) |

^{a}See footnote of table 1.
^{b}Isolated yields.
^{c}The cis:trans ratio determined by NMR.
^{d}The trans isomer ee was determined by chiral HPLC.
^{e}Sign of optical rotation.
^{f}In CH₂Cl₂ at room temperature for 24 hours using 1 mol % of [Co(P6)].
^{g}In CH₂Cl₂ at −20° C. for 48 hours using 1 mol % of [Co(P6)].
^{h}In ClC₆H₅ at room temperature for 24 hours using 2 mol % of [Co(P6)].
^{j}After one recrystallization.
^{k}[1R,2S] absolute configuration; see Table 1.
^{l}Ms: 4-methoxybenzenesulfonyl; Ns: 4-nitrobenzenesulfonyl.

In summary, we have designed and synthesized a new chiral porphyrin P6 with enhanced rigidity and polarity of chiral environment as a result of both intramolecular hydrogen bonding interactions and the use of cyclic structures. With P6 as a supporting ligand, we have demonstrated that [Co(P6)] is a highly effective catalyst for asymmetric olefin cyclopropanation with diazosulfones. The new catalytic system is general and can be applied to various aromatic olefins as well as electron-deficient olefins, leading to high-yielding formations of the corresponding cyclopropyl sulfones in both high diastereoselectivity and high enantioselectivity. Furthermore, the [Co(P6)]-based asymmetric cyclopropanation can be operated effectively in a one-pot fashion with olefins as limiting reagents and requires no slow addition of diazo reagents. This practical protocol is atypical for many other catalytic cyclopropanation systems, due to the competitive carbene dimerization side reaction, but is a common feature for [Co(Por)]-catalyzed cyclopropanation.

### DEFINITIONS

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

The term “heteroatom” shall mean atoms other than carbon and hydrogen.

The “heterosubstituted methyl” moieties described herein are methyl groups in which the carbon atom is covalently bonded to at least one heteroatom and optionally with hydrogen, the heteroatom being, for example, a nitrogen, oxygen, silicon, phosphorous, boron, sulfur, or halogen atom. The heteroatom may, in turn, be substituted with other atoms to form a heterocyclo, alkoxy, alkenoxy, alkynoxy, aryloxy, hydroxy, protected hydroxy, oxy, acyloxy, nitro, amino, amido, thiol, ketals, acetals, esters or ether moiety.

The “heterosubstituted acetate” moieties described herein are acetate groups in which the carbon of the methyl group is covalently bonded to at least one heteroatom and optionally with hydrogen, the heteroatom being, for example, a nitrogen, oxygen, silicon, phosphorous, boron, sulfur, or halogen atom. The heteroatom may, in turn, be substituted with other atoms to form a heterocyclo, alkoxy, alkenoxy, alkynoxy, aryloxy, hydroxy, protected hydroxy, oxy, acyloxy, nitro, amino, amido, thiol, ketals, acetals, esters or ether moiety.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The terms “halogen” or “halo” as used herein alone or as part of another group refer to chlorine, bromine, fluorine, and iodine.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The term “heteroaromatic” as used herein alone or as part of another group denote optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The term “acyl,” as used herein alone or as part of another group, denotes the moiety formed by removal of the hydroxyl group from the group —COOH of an organic carboxylic acid, e.g., RC(O)—, wherein R is R¹, R¹O—, R¹R²N—, or R¹S—, R¹ is hydrocarbyl, heterosubstituted hydrocarbyl, or heterocyclo and R² is hydrogen, hydrocarbyl or substituted hydrocarbyl.

The term “acyloxy,” as used herein alone or as part of another group, denotes an acyl group as described above bonded through an oxygen linkage (—O—), e.g., RC(O)O— wherein R is as defined in connection with the term “acyl”.

Unless otherwise indicated, the alkoxycarbonyloxy moieties described herein comprise lower hydrocarbon or substituted hydrocarbon or substituted hydrocarbon moieties.

The term porphyrin refers to a compound comprising a fundamental skeleton of four pyrrole nuclei united through the α-positions by four methane groups to form the following macrocyclic structure:

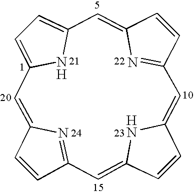

Unless otherwise indicated, the carbamoyloxy moieties described herein are derivatives of carbamic acid in which one or both of the amine hydrogens is optionally replaced by a hydrocarbyl, substituted hydrocarbyl or heterocyclo moiety.

### EXAMPLES

General Considerations: All reactions were carried out under a nitrogen atmosphere in oven-dried glassware following standard Schlenk techniques. Tetrahydrofuran (THF), and toluene were distilled under nitrogen from sodium benzophenone ketyl prior to use. Chlorobenzene was distilled under nitrogen from calcium hydride. Chiral amides purchased from Aldrich Chemical Company. and Acros Organics were used without further purification. Anhydrous cobalt (II) chloride, cobalt acetate tetrahydrate, palladium (II) acetate, and 9,9-dimethyl-4,5-bis(diphenylphosphino)xanthene (Xantphos) were purchased from Strem Chemical Company. Cesium carbonate was obtained as a gift from Chemetall Chemical Products, Incorporated. Neutral aluminum oxide powder (activated, neutral, Brockmann I) was purchased from Sigma-Aldrich company. 1-Diazo-1-(toluene-4-sulfonyl)-propan-2-one, 1-diazo-1-(4-methoxy-benzenesulfonyl)-propan-2-one, and 1-diazo-1-(4-nitrobenzesulfonyl)-propan-2-one were synthesized using typical diazo transfer reaction conditions with p-acetamidobenzenesulfonyl azide (ABSA) as the diazo transfer reagent. (Davies et al., J. Org. Chem., 1995, 60, 7529; Hodson et al., J. Chem. Soc. C, 1968, 2201). Thin layer chromatography was performed on Merck TLC plates (silica gel 60 F254). Flash column chromatography was performed with ICN silica gel (60 Å, 230-400 mesh, 32-63 μm). Proton and carbon nuclear magnetic resonance spectra (¹H NMR and ¹³C NMR) were recorded on a Varian Mercury 400 spectrometer or Bruker 250-MHz instrument and referenced with respect to internal TMS standard. HPLC measurements were carried out on a Shimadzu HPLC system with Whelk-O 1 or Chiralcel OD-H column. Infrared spectra were measured with a Nicolet Avatar 320 spectrometer with a Smart Miracle accessory. HRMS data was obtained on an Agilent 1100 LC/MS ESI/TOF mass spectrometer with electrospray ionization.

TABLE S1
Reaction results of free chiral porphyrin and Co(II) porphyrin synthesis.
Scheme S1

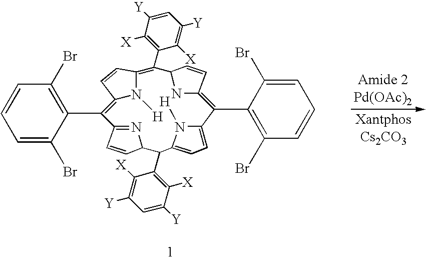

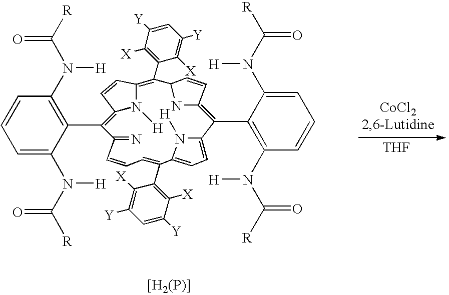

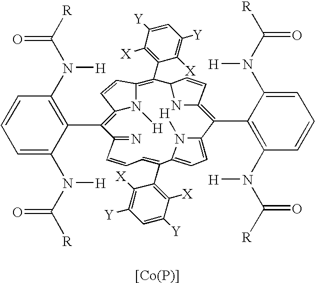

| Bromoporphyrin 1 | Amide 2 | [H₂(P)]: yield | [Co(P)]: yield |
|---|---|---|---|
| 1a (X = H; Y = t- Bu) | 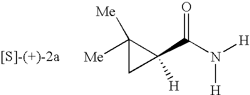 | P1: 85% | [Co(P1)]: 91% |
| 1b (X = OMe; Y = | | P2: 59% | [Co(P2)]: 95% |
| H) | | | |
| 1a (X = H; Y = t- Bu) | 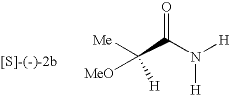 | P3: 72% | [Co(P3)]: 92% |
| 1b (X = OMe; Y = | | P4: 63% | [Co(P4)]: 95% |
| H) | | | |
| 1a (X = H; Y = t- Bu) | 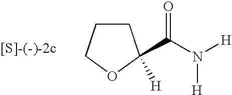 | P5: 63% | [Co(P5)]: 91% |
| 1b (X = OMe; Y = | | P6: 60% | [Co(P6)]: 89% |
| H) | | | |

General Procedures for Amidation of Bromoporphyrin. (Chen et al., J. Am. Chem. Soc. 2004, 126, 14718.) The bromoporphyrin 1, chiral amide 2, Pd(OAc)₂, Xantphos, and Cs₂CO₃ were placed in an oven-dried, resealable Schlenk tube. The tube was capped with a Teflon screwcap, evacuated, and backfilled with nitrogen. The screwcap was replaced with a rubber septum, and THF was added via syringe. The tube was purged with nitrogen for 2 min, and then the septum was replaced with the Teflon screwcap. The tube was sealed, and its contents were heated with stirring. The resulting mixture was cooled to room temperature, taken up in ethyl acetate and concentrated in vacuo. The crude product was then purified by flash chromatography.

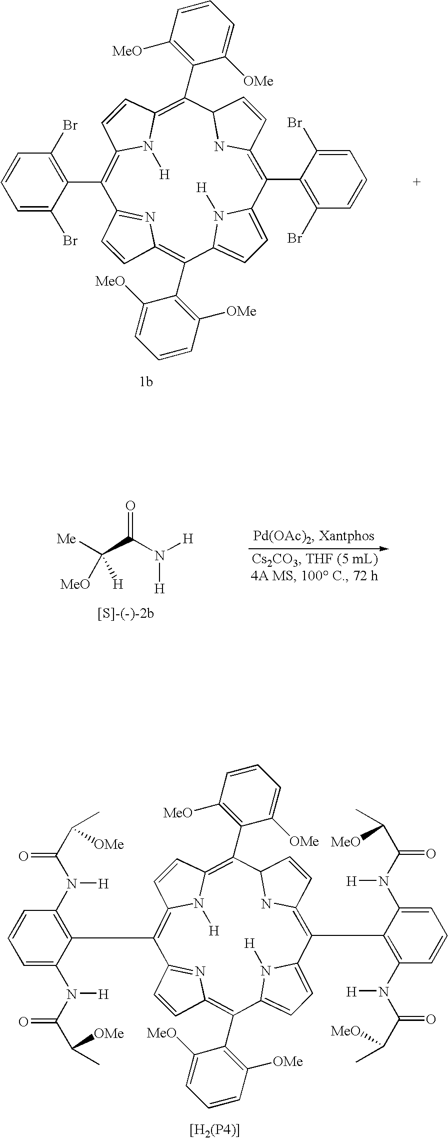

Synthesis of free porphyrin [H₂(P4)]: The general procedure was used to couple 5,15-bis(2,6-dibromophenyl)-10,20-bis(2,6-dimethoxyphenyl)-porphyrin 1b (0.105 g, 0.1 mmol) with (S)-(−)-2-methoxypropionamide 2b (0.165 g, 1.6 mmol), using Pd(OAc)₂ (0.009 g, 0.04 mmol), Xantphos (0.046 g, 0.08 mmol), and Cs₂CO₃ (0.527 g, 1.6 mmol). The reaction was conducted in THF (5 mL) at 100° C. for 72 h. The pure compound was isolated by flash column chromatography (silica gel, ethyl acetate:hexanes (v/v)=2:1) as purple solid (0.072 g, 63%). ¹H NMR (400 MHz, CDC1₃): [symbol] 8.75 (d, J=4.4 Hz, 4H), 8.69 (d, J=4.0 Hz, 4H), 8.53 (d, J=8.4 Hz, 4H), 7.86 (t, J=8.4 Hz, 2H), 7.81 (s, 4H, Amide-H), 7.76 (t, J=8.4 Hz, 2H), 7.01 (d, J=8.8 Hz, 4H), 3.51 (s, 12H), 3.04 (q, J=6.8 Hz, 4H), 1.25 (s, 12H), 0.68 (d, J=6.8 Hz, 12H), −2.42 (s, 2H). ¹³C NMR (100 MHz, CDCl₃): [symbol] 171.4, 160.3, 138.5, 130.8, 130.4, 122.6, 118.5, 117.1, 113.5, 106.8, 104.1, 78.1, 56.0, 55.8, 17.8. IR (neat, cm⁻¹): 3363, 2925, 1693, 1586, 1468, 1106. UV-Vis (CHCl₃), λₘₐₓ nm (log ε): 422 (4.74), 515 (3.96), 545 (3.36), 592 (3.52), 646 (3.29). HRMS (ESI) ([M+H]⁺) Calcd. for C₆₄H₆₇N₈O₁₂: 1139.4878. Found 1139.4857.

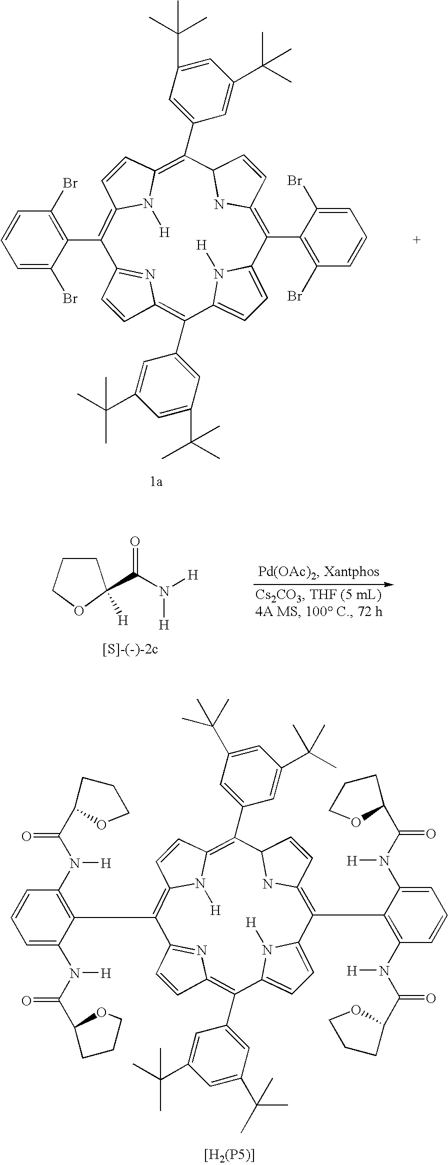

Synthesis of free porphyrin [H₂(P5)]: The general procedure was used to couple 5,15-bis(2,6-dibromophenyl)-10,20-bis(3,5-di-t-butylphenyl)porphyrin 1a (0.115 g, 0.1 mmol) with (S)-(−)-2-tetrahydrofuran-2-carboxylic acid amide 2c (0.184 g, 1.6 mmol), using Pd(OAc)₂ (0.009 g, 0.04 mmol), Xantphos (0.046 g, 0.08 mmol), and Cs₂CO₃ (0.527 g, 1.6 mmol). The reaction was conducted in THF (5 mL) at 100° C. for 72 h. The pure compound was isolated by flash column chromatography (silica gel, ethyl acetate:hexanes (v/v)=1:3) as purple solid (0.081 g, 63%). ¹H NMR (250 MHz, CDC1₃): [symbol] 8.82 (d, J=4.8 Hz, 4H), 8.71 (d, J=4.8 Hz, 4H), 8.52 (d, J=8.3 Hz, 4H), 7.88 (s, 4H, Amide-H), 7.77-7.87 (m, 8H), 7.20-7.21 (m, 4H), 3.65 (dd, J=4.5 Hz, 4H), 1.61-1.65 (m, 8H), 1.45 (s, 36H), 0.82-0.85 (m, 8H), 0.51-0.55 (m, 4H), 0.22-0.66 (m, 4H), 2.56 (s, 2H). ¹³C NMR (100 MHz, CDCl₃): [symbol] 171.5, 149.5, 140.2, 138.6, 133.4, 131.0, 129.9, 122.8, 121.9, 116.7, 108.3, 78.0, 67.9, 35.3, 31.9, 29.9, 24.4. IR (neat, cm⁻¹): 3348, 2967, 1699, 1587, 1469, 1106. UV-Vis (CHCl₃), λₘₐₓ nm (log ε): 422 (4.90), 516 (3.92), 552 (3.52), 592 (3.49), 647 (3.37). HRMS (ESI) ([M+H]⁺) Calcd. for C₈₀H₉₁N₈O₁₂: 1291.6960. Found 1291.6973.

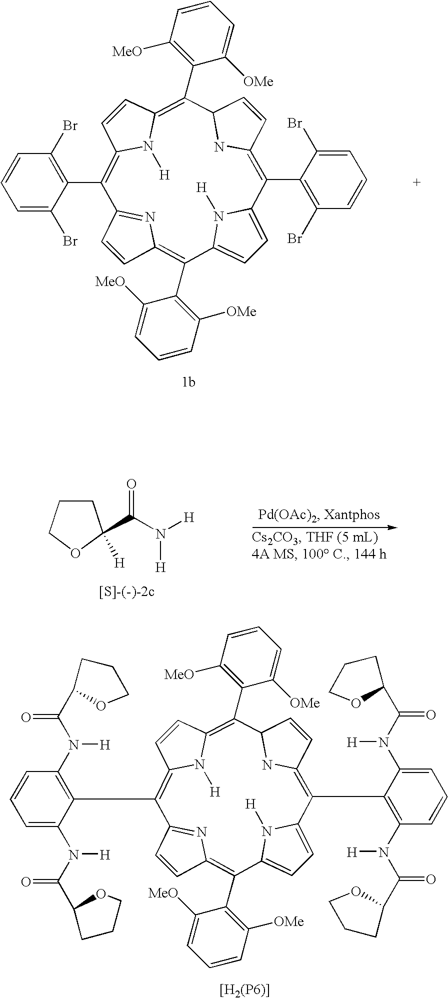

Synthesis of free porphyrin [H₂(P6)]: The general procedure was used to couple 5,15-bis(2,6-dibromophenyl)-10,20-bis(2,6-dimethoxyphenyl)-porphyrin 1b (0.105 g, 0.1 mmol) with (S)-(−)-2-tetrahydrofuran-2-carboxylic acid amide 2c (0.184 g, 1.6 mmol), using Pd(OAc)₂ (0.009 g, 0.04 mmol), Xantphos (0.046 g, 0.08 mmol), and Cs₂CO₃ (0.527 g, 1.6 mmol). The reaction was conducted in THF (5 mL) at 100° C. for 144 h. The pure compound was isolated by flash column chromatography (silica gel, ethyl acetate:hexanes (v/v)=4:1) as purple solid (0.072 g, 60%). ¹H NMR (250 MHz, CDC1₃): [symbol] 8.67 (d, J=4.8 Hz, 4H), 8.58 (d, J=4.8 Hz, 4H), 8.50 (d, J=8.3 Hz, 4H), 7.92 (s, 4H, Amide-H), 7.77 (t, J=8.0 Hz, 2H), 7.68 (t, J=8.0 Hz, 2H), 6.93 (d, J=8.5 Hz, 4H), 3.64 (t, J=6.5 Hz, 4H), 3.44 (s, 12H), 1.51-1.63 (m, 8H), 0.72-0.77 (m, 4H), 0.34-0.49 (m, 8H), 2.51 (s, 2H). ¹³C NMR (100 MHz, CDCl₃): [symbol] 171.7, 160.5, 138.7, 132.3, 131.0, 130.7, 122.1, 118.5, 116.7, 113.9, 107.2, 104.2, 78.0, 68.0, 55.9, 30.0, 24.3. IR (neat, cm⁻¹): 3348, 2967, 1693, 1587, 1468, 1108. UV-Vis (CHCl₃), λₘₐₓ nm (log ε): 422 (4.79), 514 (3.95), 546 (3.35), 591 (3.50), 646 (3.23). HRMS (ESI) ([M+H]⁺) Calcd. for C₆₈H₆₇N₈O₁₂: 1187.4878. Found 1187.4888.

X-Ray data for porphyrin [H₂(P6)]: The X-ray intensities were measured using Bruker-AXS SMART APEX/CCD diffractometer (MoKα, λ=0.71073 Å). Indexing was performed using SMART v5.625. Frames were integrated with SaintPlus 6.01 software package. Absorption correction was performed by multi-scan method implemented in SADABS. The structure was solved using SHELXS-97 and refined using SHELXL-97 contained in SHELXTL v6.10 and WinGX v1.70.01 programs packages. All non-hydrogen atoms were refined anisotropically. Hydrogen atoms were placed in geometrically calculated positions and included in the refinement process using riding model. H1 and H2 hydrogen atoms were found in the Fourier map and were included in the refinement process with site occupancies equal to 0.5. Because of the weak anomalous dispersion effects in diffraction measurements on the crystal (unreliable Flack parameter) the absolute configuration of the enantiomer (and absolute structure) has been assigned by the reference to an unchanging chiral center in the synthetic procedure. Crystal data and refinement conditions are shown in Table S2.

TABLE S2
Crystal data and structure refinement for porphyrin [H₂(P6)].

| | |
|---|---|
| Empirical formula | C68H66N8O12 |
| Formula weight | 1187.29 |
| Temperature | 100(2) K |
| Wavelength | 0.71073 Å |
| Crystal system, space group | Orthorhombic, C2221 |
| Unit cell dimensions | a = 10.361(2) Å; b = 25.392(5) Å; |
| | c = 2.380(4) Å |
| Volume | 5887.8(19) Å³ |
| Z, Calculated density | 4, 1.339 Mg/m³ |
| Absorption coefficient | 0.093 mm⁻¹ |
| F(000) | 2504 |
| Crystal size | 0.60 × 0.50 × 0.40 mm |
| Theta range for data collection | 1.60 to 28.3° |
| Limiting indices | −13 <= h <= 13, −32 <= k <= 33, |
| | −29 <= l <= 29 |
| Reflections collected/observed/ | 34062/3513/3948 [R(int) = 0.0498] |
| unique | |
| Completeness to theta = 28.31 | 98% |
| Absorption correction | Semi-empirical from equivalents |
| Max. and min. transmission | 0.9637 and 0.9463 |
| Refinement method | Full-matrix least-squares on F² |
| Data/restraints/parameters | 3948/0/416 |
| Goodness-of-fit on F² | 1.096 |
| Final R indices [I > 2sigma(I)] | R1 = 0.0418, wR2 = 0.0930 |
| R indices (all data) | R1 = 0.0500, wR2 = 0.0964 |
| Largest diff. peak and hole | 0.655 and −0.183 e · A⁻³ |

General Procedure for Synthesis of Cobalt Porphyrin Complex. (Chen et al., J. Am. Chem. Soc. 2004, 126, 14718.) Free-base porphyrin, anhydrous CoCl₂ were placed in an oven-dried, resealable Schlenk tube. The tube was capped with Teflon screwcap, evacuated, and backfilled with nitrogen. The screwcap was replaced with a rubber septum, then dry THF, and 2,6-lutidine were added via syringe. The tube was purged with nitrogen for 2 min, and then the septum was replaced with the Teflon screwcap. The tube was sealed, and its contents were heated with stirring.

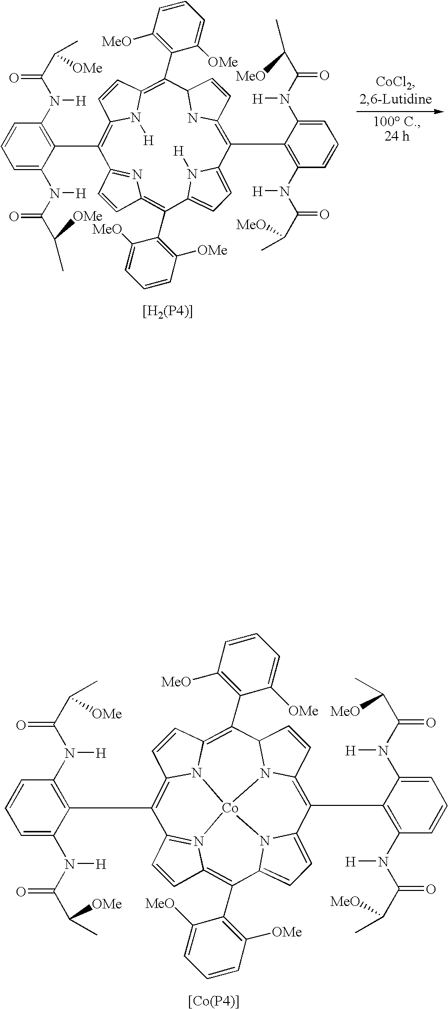

Synthesis of cobalt porphyrin [Co(P4)]: The general procedure for synthesis of cobalt porphyrin complex. Free-base porphyrin [H₂(P4)] (0.1 mmol), anhydrous CoCl₂ (0.8 mmol), 2,6-lutidine (0.4 mmol), and dry THF (5 ml) were heated at 100° C. under N₂ for 24 h. The resulting mixture was cooled to room temperature, taken up in ethyl acetate and transferred to a separatory funnel. The mixture was washed with water 3 times and concentrated in vacuo. The pure compound was obtained after flash column chromatography (silica gel, ethyl acetate:hexanes (v/v)=2:1) as a red solid (95%). IR (neat, cm⁻¹): 3367, 2925, 1693, 1587, 1469, 1107. UV-Vis (CHCl₃), λₘₐₓ nm (log ε): 412 (4.76), 528 (3.86). HRMS (ESI) ([M+H]⁺) Calcd. for C₆₄H₆₅CoN₈O₁₂: 1196.4054. Found 1196.4042.

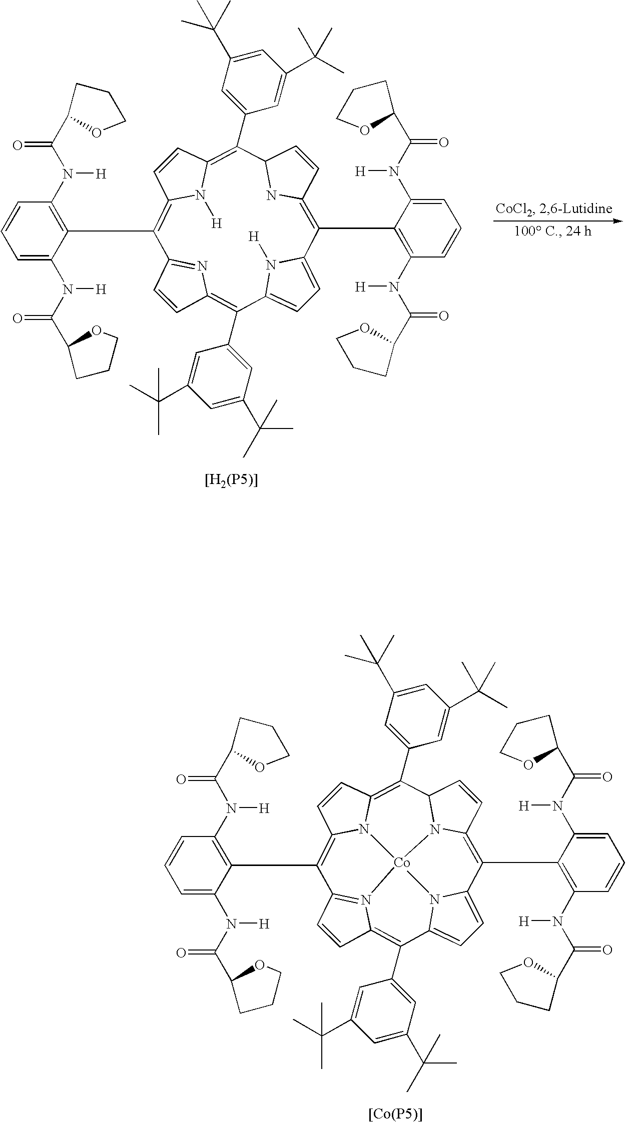

Synthesis of cobalt porphyrin [Co(P5)]: The general procedure for synthesis of cobalt porphyrin complex. Free-base porphyrin [H₂(P5)] (0.1 mmol), anhydrous CoCl₂ (0.8 mmol), 2,6-lutidine (0.4 mmol), and dry THF (5 ml) were heated at 100° C. under N₂ for 24 h. The resulting mixture was cooled to room temperature, taken up in ethyl acetate and transferred to a separatory funnel. The mixture was washed with water 3 times and concentrated in vacuo. The pure compound was obtained after flash column chromatography (silica gel, ethyl acetate:hexanes (v/v)=2:1) as a red solid (91%). IR (neat, cm⁻¹): 3348, 2959, 1693, 1587, 1468, 1106. UV-Vis (CHCl₃), λₘₐₓ nm (log ε): 415 (4.70), 529 (3.81). HRMS (ESI) ([M+H]⁺) Calcd. for C₈₀H₈₈CoN₈O₈: 1348.6135. Found 1348.6087.

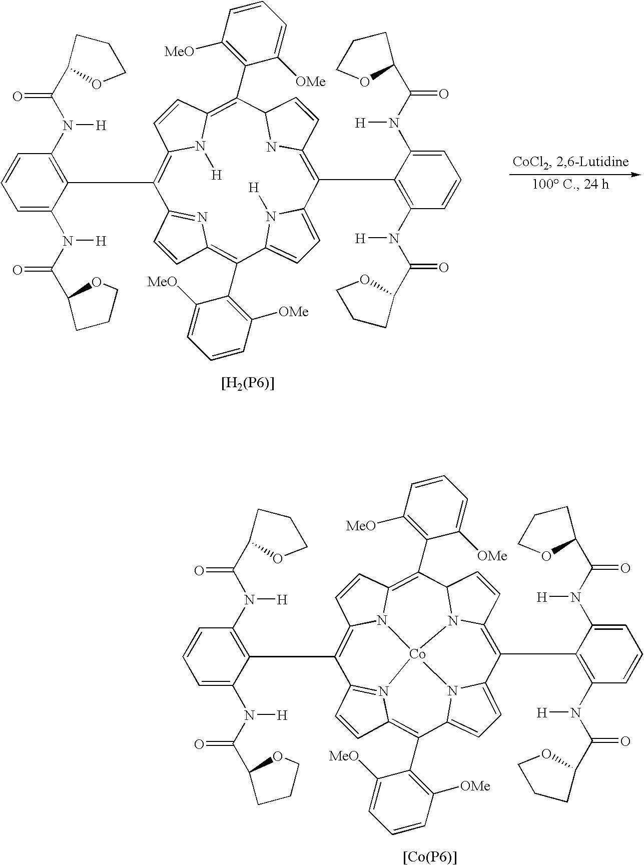

Synthesis of cobalt porphyrin [Co(P6)]: The general procedure for synthesis of cobalt porphyrin complex. Free-base porphyrin [H₂(P6)] (0.1 mmol), anhydrous CoCl₂ (0.8 mmol), 2,6-lutidine (0.4 mmol), and dry THF (5 ml) were heated at 100° C. under N₂ for 24 h. The resulting mixture was cooled to room temperature, taken up in ethyl acetate and transferred to a separatory funnel. The mixture was washed with water 3 times and concentrated in vacuo. The pure compound was obtained after flash column chromatography (silica gel, ethyl acetate:hexanes (v/v)=4:1) as a red solid (89%). IR (neat, cm⁻¹): 3347, 2966, 1692, 1587, 1468, 1108. UV-Vis (CHCl₃), λₘₐₓ nm (log ε): 412 (4.84), 529 (3.84). HRMS (ESI) ([M+H]⁺) Calcd. for C₆₈H₆₅CoN₈O₁₂: 1244.4054. Found 1244.4016.

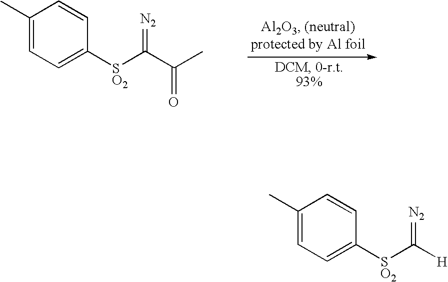

Synthesis of 1-diazomethanesulfonyl-4-methyl-benzene: To a well-stirred suspension of Al₂O₃ (100 g) in anhydrous methylene chloride (200 ml) at 0° C. protected from light by aluminum foil, 1-diazo-1-(toluene-4-sulfonyl)-propan-2-one (2.38 g, 10 mmol) was added under 0° C. and left in the ice bath to slowly rise to room temperature. The reaction was monitored by TLC every half hour until all the starting material had been consumed. The reaction mixture was then poured into an empty flash chromatography column and the alumina was washed with methylene chloride until all the product was washed out. The product was collected and concentrated by rotary evaporation at room temperature to give the pure title compound as a yellow solid (1.82 g, 93%) (van Leusen et al., Recl. Tray. Chim. Pays-Bas, 1965, 84, 151.) The product was shielded from light with aluminum foil and stored at −20° C. until used. ¹H NMR (250 MHz, CDCl₃): δ 7.69 (d, J=8.3 Hz, 2H), 7.27 (d, J=8.3 Hz, 2H), 5.20 (s, 1H), 2.38 (s, 3H). ¹³C NMR (62.5 MHz, CDCl₃): δ 144.4, 141.3, 130.0, 126.3, 57.8, 21.7. IR (neat, cm⁻¹): 3070, 2107, 1595, 1330, 1151, 660.

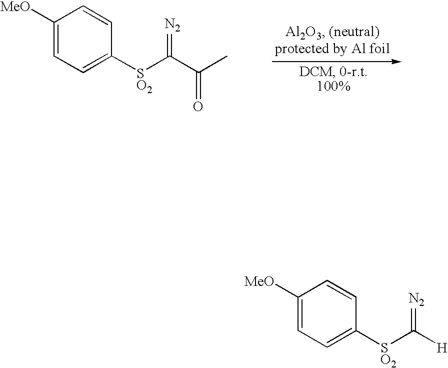

Synthesis of 1-diazomethanesulfonyl-4-methoxybenzene: To a well-stirred suspension of Al₂O₃ (100 g) in anhydrous methylene chloride (200 ml) at 0° C. protected from light by aluminum foil, 1-diazo-1-(4-methoxybenzenesulfonyl)-propan-2-one^{1,2} (2.6 g, 10 mmol) was added under 0° C. and left in the ice bath to slowly rise to room temperature. The reaction was monitored by TLC every half hour until all the starting material had been consumed. The reaction mixture was then poured into an empty flash chromatography column and the alumina was washed with methylene chloride until all the product was washed out. The product was collected and concentrated by rotary evaporation at room temperature to give the pure title compound³ as a yellow solid (2.1 g, 10 mmol). Yield=100%. The product was shielded from light with aluminum foil and stored at −20° C. until used. ¹H NMR (250 MHz, CDCl₃): δ 7.75 (d, J=9.0 Hz, 2H), 6.93 (d, J=9.0 Hz, 2H), δ 5.19 (s, 1H), δ 3.82 (s, 3H). IR (neat, cm⁻¹): 2125, 1327, 1135, 665.

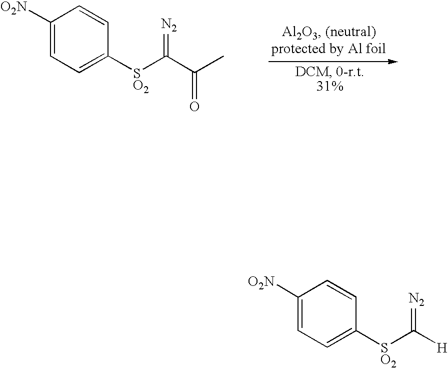

Synthesis of 1-diazomethanesulfonyl-4-nitrol-benzene: To a well-stirred suspension of Al₂O₃ (18 g) in anhydrous methylene chloride (36 ml) at 0° C. protected from light by aluminum foil, 1-diazo-1-(4-nitro-benzenesulfonyl)-propan-2-one (0.48 g, 1.78 mmol) was added under 0° C. and left in the ice bath to slowly rise to room temperature. The reaction was monitored by TLC every half hour until all the starting material had been consumed. The reaction mixture was then poured into an empty flash chromatography column and the alumina was washed with methylene chloride until all the product was washed out. The product was collected and concentrated by rotary evaporation at room temperature to give the pure title compound as a yellow solid (0.125 g, 0.55 mmol). Yield=31%. The product was shielded from light with aluminum foil and stored at −20° C. until used. ¹H NMR (250 MHz, CDCl₃): δ 8.34 (d, J=9.0 Hz, 2H), 8.01 (d, J=9.0 Hz, 2H), 5.29 (s, 1H). IR (neat, cm⁻¹): 2117, 1522, 1150, 612.

General Procedures for Cyclopropanation of Styrene. Catalyst (1 mol %) was placed in an oven-dried, resealable Schlenk tube. The tube was capped with a Teflon screwcap, evacuated, and backfilled with nitrogen. The screwcap was replaced with a rubber septum, and 1.0 equivalent of styrene (0.25 mmol) in 0.5 mL DCM was added via syringe, followed by 1.2 equivalents of diazo compound, and followed by the remaining DCM (0.5 mL). The tube was purged with nitrogen for 1 min and its contents were stirred at room temperature. After the reaction finished, the resulting mixture was concentrated and the residue was purified by flash silica gel chromatography to give the product.

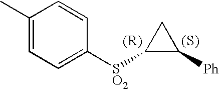

1-Methyl-4-(2-phenylcyclopropylsulfonyl)benzene: (Bellesia et al., J. Chem. Res. Miniprint 1981, 4, 1301; Balaji, R. Indian J. Chem. Sect. B 1979, 18, 454.) Trans-isomer: [α]²⁰_{D}=−31.4 (c=0.32, CHCl₃). ¹H NMR (400 MHz, CDCl₃): δ 7.80 (d, J=7.6 Hz, 2H), 7.35 (d, J=8.0 Hz, 2H), 7.19-7.25 (m, 3H), 7.00-7.02 (m, 2H), 2.84-2.89 (m, 1H), 2.61-2.66 (m, 1H), 2.44 (s, 3H), 1.84-1.89 (m, 1H), 1.42-1.47 (m, 1H). ¹³C NMR (100 MHz, CDCl₃): δ 144.6, 137.8, 137.8, 130.2, 128.8, 127.8, 127.3, 126.8, 42.1, 24.0, 21.9, 14.1. IR (neat, cm⁻¹): 2925, 1602, 1457, 1301, 1141, 697. HRMS (ESI) ([M+H]⁺) Calcd. for C₁₆H₁₇O₂S: 273.0949. Found 273.0936. HPLC analysis: ee=92%. Whelk-O 1 (80% hexanes: 20% isopropanol, 1.0 mL/min) trans-isomer: tₘᵢₙₒᵣ=19.2 min, tₘₐⱼₒᵣ=25.1 min.

X-Ray data for 1-methyl-4-(2-phenylcyclopropyl-sulfonyl)benzene: The X-ray intensities were measured using Bruker-AXS SMART APEX/CCD diffractometer (MoKα, λ=0.71073 Å). Indexing was performed using SMART v5.625. Frames were integrated with SaintPlus 6.01 software package. Absorption correction was performed by multi-scan method implemented in SADABS. The structure was solved using SHELXS-97 and refined using SHELXL-97 contained in SHELXTL v6.10 and WinGX v1.70.01 programs packages. All non-hydrogen atoms were refined anisotropically. H8, H91, H92 and H10 hydrogen atoms were found in the Fourier map and were included in the refinement process without constraints. Hydrogen atoms of the phenyl and tolyl groups were placed in geometrically calculated positions and included in the refinement process using riding model. Absolute configuration (and absolute structure) was established by anomalous-dispersion effects in diffraction measurements on the crystal. Crystal data and refinement conditions are shown in Table S3.

TABLE S3
Crystal data and refinement for 1-methyl-
4-(2-phenylcyclopropyl-sulfonyl)benzene.

| | |
|---|---|
| Empirical formula | C16 H16 O2 S |
| Formula weight | 272.35 |
| Temperature | 133(2) K |
| Wavelength | 0.71073 Å |
| Crystal system, space group | Orthorhombic, P2(1)2(1)2(1) |
| Unit cell dimensions | a = 7.6379(17) Å; b = |
| | 11.269(3) Å; c = |
| | 15.319(3) Å |
| Volume | 1318.6(5) Å³ |
| Z, Calculated density | 4, 1.372 Mg/m³ |
| Absorption coefficient | 0.240 mm⁻¹ |
| F(000) | 576 |
| Crystal size | 0.20 × 0.10 × 0.10 mm |
| Theta range for data collection | 2.24 to 25.29° |
| Limiting indices | −9 <= h <= 9, −13 <= |
| | k <= 13, −18 <= l <= 18 |
| Reflections collected/observed/ | 12968/2332/2401 [R(int) = 0.0493] |
| unique | |
| Completeness to theta = 28.31 | 100.0% |
| Absorption correction | Semi-empirical from equivalents |
| Max. and min. transmission | 0.9764 and 0.9536 |
| Refinement method | Full-matrix least-squares on F² |
| Data/restraints/parameters | 2401/0/189 |
| Goodness-of-fit on F² | 1.037 |
| Final R indices [I > 2sigma(I)] | R1 = 0.0286, wR2 = 0.0755 |
| R indices (all data) | R1 = 0.0296, wR2 = 0.0765 |
| Absolute structure parameter | 0.01(6) |
| Largest diff. peak and hole | 0.256 and −0.189 e.A⁻³ |

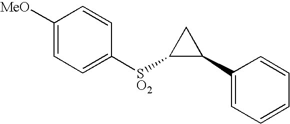

1-Methoxy-4-(2-phenylcyclopropylsulfonyl)benzene: Trans-isomer: [α]²⁰_{D}=−33.4 (c=0.68, CHCl₃). ¹H NMR (250 MHz, CDC1₃): δ 7.79 (d, J=8.8 Hz, 2H), 7.14-7.19 (m, 3H), 6.95 (d, J=8.8 Hz, 4H), 3.81 (s, 3H), 2.83-2.75 (m, 1H), 2.61-2.54 (m, 1H), 1.84-1.75 (m, 1H), 1.42-1.34 (m, 1H). ¹³C NMR (62.5 MHz, CDC1₃), δ 163.6, 137.6, 132.2, 129.8, 128.7, 127.1, 126.6, 114.6, 55.7, 42.2, 23.8, 13.9. IR (neat, cm⁻¹): 1592, 1574, 1259, 1136, 736. HRMS (ESI) ([M+NH₄]⁺) Calcd. for C₁₆H₂₀NO₃S: 306.1164. Found 306.1164. HPLC analysis: ee=96%. Whelk-O 1 (80% hexanes: 20% isopropanol, 1.0 mL/min) trans-isomer: tₘᵢₙₒᵣ=28.3 min, tₘₐⱼₒᵣ=37.4 min.

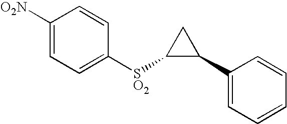

1-Nitro-4-(2-phenylcyclopropylsulfonyl)benzene: Trans-isomer: [α]²⁰_{D}=−43.5 (c=0.54, CHCl₃). ¹H NMR (250 MHz, CDC1₃): δ 8.35 (d, J=7.0 Hz, 2H), 8.07 (d, J=7.0 Hz, 2H), 7.21-7.16 (m, 3H), 6.97-6.93 (m, 2H), 2.93-2.84 (m, 1H), 2.66-2.59 (m, 1H), 1.91-1.82 (m, 1H), 1.54-1.46 (m, 1H). ¹³C NMR (62.5 MHz, CDCl₃): δ 150.7, 146.0, 136.6, 129.1, 128.9, 127.6, 126.5, 124.6, 41.5, 24.2, 14.2. IR (neat, cm⁻¹): 1592, 1259, 1138, 738. HRMS (ESI) ([M+NH₄]⁺) Calcd. for C₁₅H₁₇N₂O₄S: 321.0909. Found 321.0899. HPLC analysis: ee=90%. Whelk-O 1 (80% hexanes: 20% isopropanol, 1.0 mL/min) trans-isomer: tₘᵢₙₒᵣ=18.8 min, tₘₐⱼₒᵣ=22.2 min.

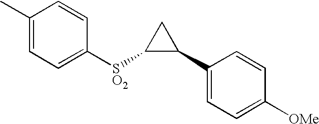

1-Methoxy-4-(2-tosylcyclopropyl)benzene: Trans-isomer: [α]²⁰_{D}=−47.1 (c=0.28, CHCl₃). ¹H NMR (250 MHz, CDC1₃): δ 7.74 (d, J=8.3 Hz, 2H), 7.28 (d, J=8.0 Hz, 2H), 6.87 (d, J=8.5 Hz, 2H), 6.70 (d, J=8.8 Hz, 2H), 3.69 (s, 3H), 2.72-2.80 (m, 1H), 2.46-2.54 (m, 1H), 2.38 (s, 3H), 1.72-1.78 (m, 1H), 1.29-1.35 (m, 1H). ¹³C NMR (62.5 MHz, CDCl₃): δ 158.8, 144.4, 140.4, 137.7, 129.9, 127.8, 127.6, 114.1, 55.3, 41.8, 23.2, 21.7, 13.7. IR (neat, cm⁻¹): 2924, 1596, 1514, 1146, 657. HRMS (ESI) ([M+H]⁺) Calcd. for C₁₇H₁₉O₃S: 303.1055. Found 303.1045. HPLC analysis: ee=95%, Whelk-O 1 (80% hexanes: 20% isopropanol, 1.0 mL/min) trans-isomer: tₘᵢₙₒᵣ=30.9 min, tₘₐⱼₒᵣ=39.6 min.

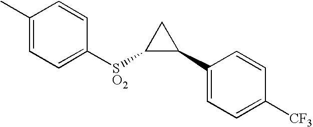

1-(2-Tosylcyclopropyl)-4-(trifluoromethyl)benzene: Trans-isomer: [α]²⁰_{D}=−38.7 (c=0.40, CHC1₃). ¹H NMR (250 MHz, CDCl₃): δ 7.74 (d, J=8.3 Hz, 2H), 7.43 (d, J=8.3 Hz, 2H), 7.29 (d, J=8.0 Hz, 2H), 7.06 (d, J=8.0 Hz, 2H), 2.80-2.89 (m, 1H), 2.58-2.65 (m, 1H), 2.39 (s, 3H), 1.81-1.89 (m, 1H), 1.37-1.45 (m, 1H). ¹³C NMR (62.5 MHz, CDCl₃): δ 158.8, 144.8, 141.8, 137.3, 130.1, 129.4, 127.7, 126.9, 125.7, 125.6, 42.2, 23.3, 21.7, 14.2. IR (neat, cm⁻¹): 2924, 1618, 1322, 1068, 831, 661. HRMS (ESI) ([M+H]⁺) Calcd. for C₁₇H₁₆F₃O₂S: 341.0823. Found 341.0831. HPLC analysis: ee=96%. Whelk-O 1 (80% hexanes: 20% isopropanol, 1.0 mL/min) trans-isomer: tₘᵢₙₒᵣ=14.9 min, tₘₐⱼₒᵣ=19.8 min.

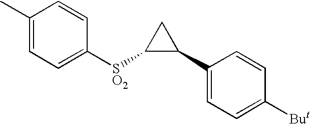

1-tert-Butyl-4-(2-tosylcyclopropyl)benzene: Trans-isomer: [α]²⁰_{D}=−46.3 (c=0.91, CHCl₃). ¹H NMR (250 MHz, CDC1₃): δ 7.73 (d, J=8.3 Hz, 2H), δ 7.27 (d, J=8.3 Hz, 2H), δ 7.19 (d, J=8.3 Hz, 2H), 6.87 (d, J=8.3 Hz, 2H), 2.72-2.80 (m, 1H), 2.52-2.59 (m, 1H), 2.36 (s, 3H), 1.74-1.82 (m, 1H), 1.31-1.39 (m, 1H), 1.19 (s, 9H). ¹³C NMR (62.5 MHz, CDCl₃): δ 150.2, 144.4, 137.7, 134.5, 130.0, 127.7, 126.4, 125.6, 41.8, 34.5, 31.3, 23.5, 21.7, 13.8. IR (neat, cm⁻¹): 2965, 1734, 1317, 1148, 831, 814, 728, 662. HRMS (ESI) ([M+H]⁺) Calcd. for C₂₀H₂₅O₂S: 329.1575. Found 329.1580. HPLC analysis: ee=94%. Whelk-O 1 (80% hexanes: 20% isopropanol, 1.0 mL/min) trans-isomer: tₘᵢₙₒᵣ=16.2 min, tₘₐⱼₒᵣ=21.5 min.

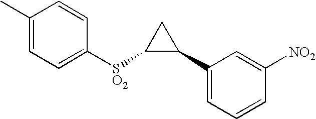

1-Nitro-3-(2-tosylcyclopropyl)benzene: Trans-isomer: [α]²⁰_{D}=−44.4 (c=0.54, CHCl₃). ¹H NMR (400 MHz, CDC1₃): δ 8.04 (d, J=6.4 Hz, 1H), 7.80 (d, J=8.0 Hz, 3H), 7.36-7.45 (m, 4H), 2.93-2.98 (m, 1H), 2.69-2.74 (m, 1H), 2.45 (s, 3H), 1.90-1.96 (m, 1H), 1.49-1.54 (m, 1H). ¹³C NMR (100 MHz, CDCl₃): δ 148.6, 145.1, 140.1, 137.3, 133.4, 130.3, 129.9, 127.9, 122.4, 121.4, 42.5, 23.2, 21.8, 14.5. IR (neat, cm⁻¹): 2924, 1528, 1349, 1146, 742, 657. HRMS (ESI) ([M+H]⁺) Calcd. for C₁₆H₁₆NO₂S: 318.0800. Found 318.0799. HPLC analysis: ee=96%. Whelk-O 1 (80% hexanes: 20% isopropanol, 1.0 mL/min) trans-isomer: tₘᵢₙₒᵣ=34.9 min, tₘₐⱼₒᵣ=47.6 min.

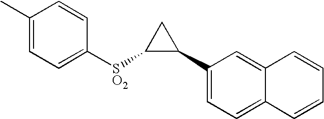

2-(2-Tosylcyclopropyl)naphthalene: Trans-isomer: [α]²⁰_{D}=−34.0 (c=0.15, CHCl₃). ¹H NMR (250 MHz, CDC1₃): δ 7.77 (d, J=8.3 Hz, 2H), 7.64-7.72 (m, 3H), 7.28-7.43 (m, 5H), 7.03 (d, J=8.5 Hz, 1H), 2.93-3.01 (m, 1H), 2.64-2.71 (m, 1H), 2.38 (s, 3H), 1.84-1.92 (m, 1H), 1.52-1.55 (m, 1H). ¹³C NMR (100 MHz, CDCl₃): δ 144.7, 137.8, 135.1, 133.4, 132.6, 130.1, 128.6, 127.8, 127.8, 127.6, 126.6, 126.1, 125.5, 42.2, 24.2, 21.8, 14.2. IR (neat, cm⁻¹): 2924, 1621, 1325, 1143, 660. HRMS (ESI) ([M+H]⁺) Calcd. for C₂₀H₁₉O₂S: 323.1106. Found 323.1097. HPLC analysis: ee=93%. Whelk-O 1 (80% hexanes: 20% isopropanol, 1.0 mL/min) trans-isomer: tₘᵢₙₒᵣ=36.7 min, tₘₐⱼₒᵣ=49.7 min.

General Procedures for Cyclopropanation of Methylacrylate. Catalyst (2 mol %) was placed in an oven-dried, resealable Schlenk tube. The tube was capped with a Teflon screwcap, evacuated, and backfilled with nitrogen. The screwcap was replaced with a rubber septum, and 1.0 equivalent of styrene (0.25 mmol) in 0.5 mL chlorobenzene was added via syringe, followed by 1.2 equivalents of diazo compound, followed by the remaining chlorobenzene (0.5 mL). The tube was purged with nitrogen for 1 min and its contents were stirred at room temperature. After the reaction finished, the resulting mixture was concentrated and the residue was purified by flash silica gel chromatography to give the product.

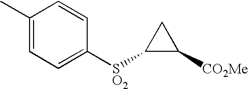

Methyl 2-tosylcyclopropanecarboxylate: Trans-isomer: [α]²⁰_{D}=−46.1 (c=0.40, CHCl₃). ¹H NMR (400 MHz, CDC1₃): δ 7.74 (d, J=8.0 Hz, 2H), 7.34 (d, J=8.0 Hz, 2H), 3.65 (s, 3H), 2.92-2.96 (m, 1H), 2.45-2.50 (m, 1H), 2.43 (s, 3H), 1.67-1.72 (m, 1H), 1.49-1.54 (m, 1H). ¹³C NMR (100 MHz, CDCl₃): δ 170.9, 145.2, 137.0, 130.3, 128.0, 52.7, 40.7, 21.9, 20.1, 13.5. IR (neat, cm⁻¹): 2924, 1732, 1148, 716. HRMS (ESI) ([M+H]⁺) Calcd. for C₁₂H₁₅O₄S: 255.0691. Found 255.0668. HPLC analysis: ee=90%. Chiralcel OD-H (98% hexanes: 2% isopropanol, 1.0 mL/min) trans-isomer: tₘᵢₙₒᵣ=29.4 min, tₘₐⱼₒᵣ=35.3 min.

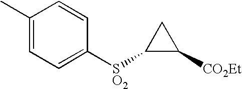

Ethyl 2-tosylcyclopropanecarboxylate: Trans-isomer: [α]²⁰_{D}=−38.2 (c=0.49, CHCl₃). ¹H NMR (400 MHz, CDC1₃): δ 7.75 (d, J=8.0 Hz, 2H), 7.34 (d, J=8.0 Hz, 2H), 4.10 (q, J=7.2 Hz, 2H), 2.91-2.96 (m, 1H), 2.45-2.50 (m, 1H), 2.44 (s, 3H), 1.65-1.70 (m, 1H), 1.48-1.53 (m, 1H), 1.22 (t, J=7.2 Hz, 3H). ¹³C NMR (100 MHz, CDC1₃): δ 170.5, 145.1, 137.0, 130.2, 128.0, 61.8, 40.6, 21.9, 20.3, 14.3, 13.6. IR (neat, cm⁻¹): 2919, 1729, 1149, 716. HRMS (ESI) ([M+H]⁺) Calcd. for C₁₃H₁₇O₄S: 269.0848. Found 269.0849. HPLC analysis: ee=90%. Chiralcel OD-H (99.3% hexanes: 0.7% isopropanol, 2.0 mL/min) trans-isomer: tₘᵢₙₒᵣ=63.4 min, tₘₐⱼₒᵣ=79.5 min.

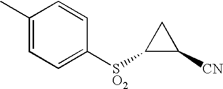

2-Tosylcyclopropanecarbonitrile: Trans-isomer: [α]²⁰_{D}=−28.4 (c=0.29, CHCl₃). ¹H NMR (400 MHz, CDC1₃): δ 7.74 (d, J=8.4 Hz, 2H), 7.38 (d, J=8.4 Hz, 2H), 3.00-3.05 (m, 1H), 2.46 (s, 3H), 2.20-2.24 (m, 1H), 1.80-1.86 (m, 1H), 1.59-1.64 (m, 1H). ¹³C NMR (100 MHz, CDCl₃): δ 146.0, 135.9, 130.6, 128.1, 117.7, 39.3, 21.9, 12.8, 4.8. IR (neat, cm⁻¹): 2248, 1150, 659. HRMS (ESI) ([M+H]⁺) Calcd. for C₁₁H₁₂NO₂S: 222.0589. Found 222.0572. HPLC analysis: ee=61%. Whelk-O 1 (95% hexanes: 5% isopropanol, 1.0 mL/min) trans-isomer: tₘᵢₙₒᵣ=70.5 min, tₘₐⱼₒᵣ=83.6 min.

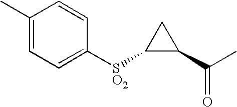

1-(2-Tosylcyclopropyl)ethanone: Trans-isomer: [α]²⁰_{D}=−91.5 (c=0.81, CHCl₃), ee=89%. ¹H NMR (400 MHz, CDC1₃): δ 7.73 (d, J=8.0 Hz, 2H), 7.33 (d, J=8.0 Hz, 2H), 2.90-2.94 (m, 1H), 2.74-2.78 (m, 1H), 2.43 (s, 3H), 2.28 (s, 3H), 1.61-1.66 (m, 1H), 1.44-1.49 (m, 1H). ¹³C NMR (100 MHz, CDCl₃): δ 203.8, 145.1, 137.0, 130.3, 127.9, 42.3, 31.3, 26.5, 21.8, 15.3. IR (neat, cm⁻¹): 1733, 1705, 1144, 732. HRMS (ESI) ([M+H]⁺) Calcd. for C₁₂H₁₅O₃S: 239.0742. Found 239.0738. HPLC analysis: Chiralcel OD-H (98% hexanes: 2% isopropanol, 1.0 mL/min) trans-isomer: tₘᵢₙₒᵣ=35.8 min, tₘₐⱼₒᵣ=39.5 min.

The foregoing non-limiting examples are provided to illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

What is claimed is:

1. A process for cyclopropanation of an olefin, the process comprising treating the olefin with a diazosulfone in the presence of a metal porphyrin complex.

2. The process of claim 1 wherein the metal porphyrin complex is a cobalt porphyrin complex.

3. The process of claim 2 wherein the olefin corresponds to Formula 1 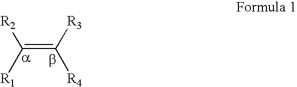
wherein R₁, R₂, R₃, and R₄ are independently hydrogen, hydrocarbyl, substituted hydrocarbyl, heterocyclo or EWG, and EWG is an electron-withdrawing group.

4. The process of claim 3 wherein at least one of R₁, R₂, R₃, and R₄ is optionally substituted alkyl, optionally substituted alkenyl, optionally substituted alkynyl, or optionally substituted aryl.

5. The process of claim 3 wherein at least one of R₁, R₂, R₃, and R₄ is phenyl, tert-butyl phenyl, methoxyphenyl, trifluoromethyl phenyl, nitrophenyl, or naphthyl.

6. The process of claim 3 wherein at least one of R₁, R₂, R₃, and R₄ is p-tert-butyl phenyl, p-methoxyphenyl, p-trifluoromethyl phenyl, 3-nitrophenyl, or naphthyl.

7. The process of claim 3 wherein at least one of R₁, R₂, R₃, and R₄ is —CN, —C(O)R₂₂, or —C(O)OR₂₂ wherein R₂₂ is optionally substituted alkyl, optionally substituted alkenyl, optionally substituted alkynyl, or optionally substituted aryl.

8. The process of claim 3 wherein at least one of R₁, R₂, R₃, and R₄ is —CN, —C(O)CH₃, —C(O)OCH₃, or —C(O)OC₂H₅.

9. The process of claim 2 wherein the olefin is an aromatic olefin, an α,β-unsaturated alkene, an α,β-unsaturated ester, an α,β-unsaturated ketone, or an α,β-unsaturated nitrile.

10. The process of claim 2 wherein the olefin is styrene or substituted styrene.

11. The process of claim 2 wherein the diazosulfone corresponds to Formula 6 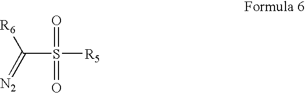
wherein R₅ and R₆ are independently hydrogen, hydrocarbyl, substituted hydrocarbyl, or heterocyclo.

12. The process of claim 11 wherein R₆ is hydrogen, alkyl or substituted alkyl, and R₅ is substituted alkyl, substituted alkenyl, substituted alkynyl, substituted phenyl, substituted aryl, or heterosubstituted phenyl.

13. The process of claim 11 wherein R₆ is hydrogen, alkyl or substituted alkyl, and R₅ is optionally substituted phenyl.

14. The process of claim 11 wherein R₆ is hydrogen and R₅ is phenyl, p-methylphenyl, p-nitrophenyl, or p-methoxyphenyl.

15. The process of claim 2 wherein the cobalt porphyrin complex is selected from the group of cobalt porphyrin complexes consisting of

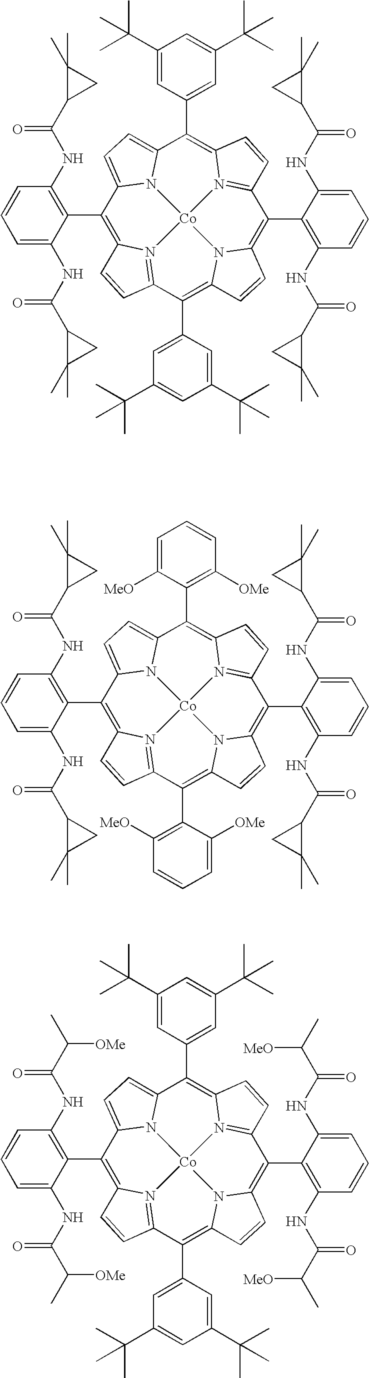

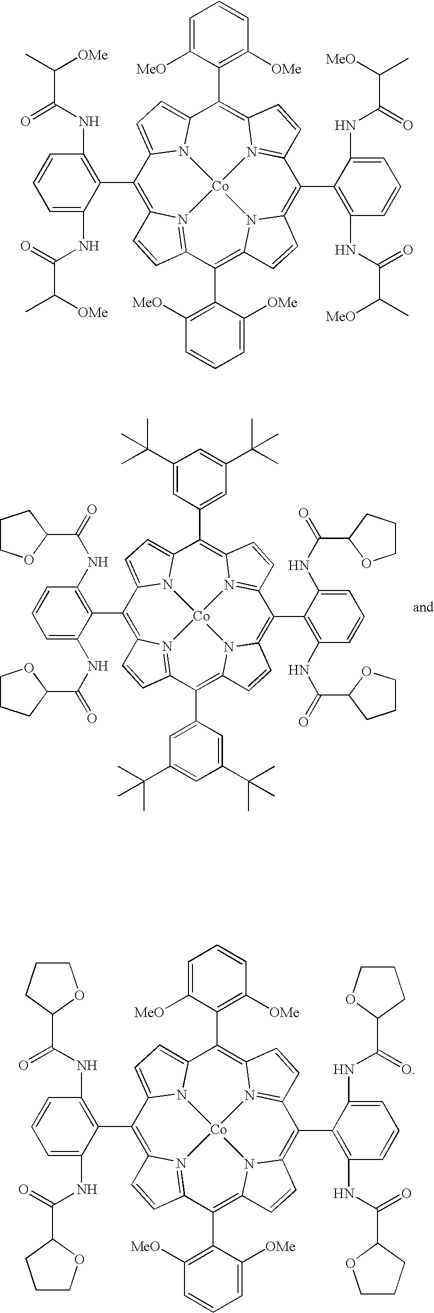

16. The process of claim 2 wherein the cobalt porphyrin complex is a cobalt (II) complex of a D₂-symmetric chiral porphyrin.

17. The process of claim 2 wherein the process yields a sulfone substituted cyclopropane corresponding to Formula A 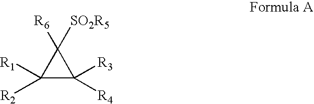
wherein R₁, R₂, R₃, R₄, are independently hydrogen, hydrocarbyl, substituted hydrocarbyl, heterocyclo, or an electron-withdrawing group, and R₅ and R₆ are independently hydrogen, hydrocarbyl, substituted hydrocarbyl, or heterocyclo.

18. The process of claim 17 wherein at least one of R₁, R₂, R₃, and R₄ is selected from the group consisting of p-tert-butyl phenyl, p-methoxyphenyl, p-trifluoromethyl phenyl, 3-nitrophenyl, naphthyl, —CN, —C(O)CH₃, —C(O)OCH₃, and —C(O)OC₂H₅.

19. The process of claim 2 wherein the process yields a sulfone substituted cyclopropane corresponding to Formula C 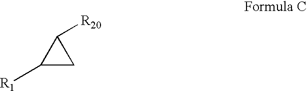
wherein R₁ is hydrogen, hydrocarbyl, substituted hydrocarbyl, heterocyclo, or an electron withdrawing group, and R₂₀ is a sulfonyl group corresponding to —SO₂R₂₄ wherein R₂₄ is hydrogen, hydrocarbyl, substituted hydrocarbyl, or heterocyclo.

20. The process of claim 19 wherein R₁ is selected from the group consisting of p-tert-butyl phenyl, p-methoxyphenyl, p-trifluoromethyl phenyl, 3-nitrophenyl, naphthyl, —CN, —C(O)CH₃, —C(O)OCH₃, and —C(O)OC₂H₅, and wherein R₂₀ is selected from the group consisting of tosyl, methoxyphenylsulfonyl, and nitrophenylsulfonyl.
